(19) United States
(12) Patent Application Publication
(10) Pub. No.: US 2002/0156018 A1
(43) Pub. Date: Oct. 24, 2002

(54) Treatment of parasitic diseases by inhibition of cysteine proteases of the papain superfamily

(76) Inventors: Scott Kevin Thompson, Phoenixville, PA (US); Daniel Frank Veber, Ambler, PA (US); Thaddeus Anthony Tomaszek, Collegeville, PA (US); David Graham Tew, Audubon, PA (US)

Correspondence Address: GLAXOSMITHKLINE, Corporate Intellectual Property - UW2220, P.O. Box 1539, King of Prussia, PA, 19406-0939, US

(21) Appl. No.: 10/120,720

(22) Filed: Apr. 12, 2002

Related U.S. Application Data
(63) Continuation of application No. 09/673,050, filed on Oct. 10, 2000, now abandoned.
Application No. 09/673,050 is a 371 of international application No. PCT/US99/07723, filed on Apr. 8, 1999.
(60) Provisional application No. 60/081,221, filed on Apr. 9, 1998.

(51) Int. Cl. A61K 38/05; A61K 31/501; A61K 31/498; A61K 31/4439

(52) U.S. Cl. 514/19; 514/247; 514/365; 514/336; 514/255.05

(57) ABSTRACT

The present invention relates to compounds and pharmaceutical compositions which inhibit proteases, such as cysteine proteases. In particular, the present invention relates to compounds and pharmaceutical compositions which inhibit cysteine proteases of the papain superfamily. The compounds and pharmaceutical compositions of the present invention are useful for treating diseases, particularly parasitic diseases, which are mediated by such proteases. In particular, the present invention relates to a method of treating malaria by inhibiting the cysteine protease falcipain.

### FIELD OF THE INVENTION

The present invention relates to methods, compounds and pharmaceutical compositions for treating malaria. In particular, the compositions comprise compounds which act as protease inhibitors which specifically inhibit cysteine proteases of the papain superfamily. The compounds of the present invention are useful for treating diseases, particularly parasitic diseases, which are mediated by the activity of such proteases. In particular, the present invention relates to treating malaria by inhibiting falcipain.

### BACKGROUND OF THE INVENTION

Infection with Plasmodium falciparum, the most virulent human malaria pathogen, infects over 280 million people and is estimated to be responsible for over I million deaths annually (Gibbons, A. Science 1992, 256, 1135; Walsh, J. A. Ann. N. Y. Acad. Sci. 1989, 569, 1135). The Plasmodium falciparum parasite has a 48 hour life cycle within host erythrocytes that is responsible for all of the clinical manifestations of falciparum malaria. During this cycle, the erythrocyte is invaded by a merozoite, then the intracellular parasite develops from a ring stage into a more metabolically active trophozoite, divides asexually and becomes a schizont, and finally ruptures the host erythrocyte, releasing daughter merozoites that invade other erythrocytes to reinitiate the cycle. During the trophozoite stage, hemoglobin from the host erythrocyte is degraded for use as the parasites principal source of amino acids.

Rosenthal and coworkers have identified a 28 kD trophozoite cysteine protease (TCP or falcipain) from malaria parasites that mediates host hemoglobin degradation (Rosenthal, P. J.; McKerrow, J. H.; Aikawa, M.; Nagasawa, H.; Leech, J. H. J. Clin. Invest. 1988, 82, 1560) and is expressed only at the trophozoite stage (Rosenthal, P. J.; Kim, J. H.; McKerrow, J. H.; Leech, J. H. J. Exp. Med. 1987, 166, 816). Inhibition of this enzyme results in a blocking of hemoglobin degradation and killing of cultured parasites (Rosenthal, P. J.; Wollish, W. S.; Palmer, J. T.; Rasnick, D. J. Clin. Invest. 1991, 88, 1467; Li, R.; Kenyon, G. L.; Cohen, F. E.; Chen, X.; Gong, B.; Dominguez, J. N.; Davidson, E.; Kurzban, G.; Miller, R. E.; Nuzum, E. O.; Rosenthal, P. J.; McKerrow, J. H. J. Med. Chem. 1995, 38, 5031). In a mouse model of infection with P. vinckei, the analogous murine malarial parasite, treatment with cysteine protease inhibitors resulted in a long-term curative effect (>75 days) in 80% of animals (Rosenthal, P. J.; Lee, G. K.; Smith R. E. J. Clin. Invest. 1993, 91, 1052). Thus, a selective inhibitor of falcipain may be an effective anti-malarial therapy either in conjunction with or as a replacement for the quinoline-derived drugs.

In addition to Plasmodium falciparum, other parasites utilize cysteine proteases in their life cycle. These include Trypanosoma cruzi, Trypanosoma Brucei [trypanosomiasis (African sleeping sickness, Chagas disease)], Leishmania mexicana, Leishmania pifanoi, Leishmania major (leishmaniasis), Schistosoma mansoni (schistosomiasis), Onchocerca volvulus [onchocerciasis (river blindness)] Brugia pahangi, Entamoeba histolytica, Giardia lamblia, the helminths, Haemonchus contortus and Fasciola hepatica, as well as helminths of the genera Spirometra, Trichinella, Necator and Ascaris, and protozoa of the genera Cryptosporidium, Eimeria, Toxoplasma and Naegleria (McKerrow, J. H. (1995) in Perspect. Drug Dis. Des. 2, eds., Craik, C. S., Debouck, C., pp. 437-444; Robertson, C. D., Coombs, G. H., North, M. J., Mottram, J. C. (1996) in Perspect. Drug Dis. Des. 6, eds., McKerrow, J. H. and James, M. N. G., pp. 99-118).

It has now been discovered that certain compounds are protease inhibitors, most particularly inhibitors of falcipain, and these compounds are useful for treating diseases caused by cysteine proteases and particularly, malaria.

### SUMMARY OF THE INVENTION

An object of the present invention is to provide protease inhibitors, such as inhibitors of cysteine proteases. In particular, the present invention relates to compounds which inhibit cysteine proteases, and particularly cysteine proteases of the papain superfamily. The compounds of the present invention are useful for treating diseases, particularly parasitic diseases, which may be therapeutically modified by altering the activity of such proteases. In particular, the present invention relates to treating malaria by inhibiting falcipain.

Accordingly, in the first aspect, this invention provides a method of treating diseases in which the disease pathology may be therapeutically modified by inhibiting proteases, such as cysteine proteases, with one or more of the following compounds:

2-[N-(N-benzyloxycarbonylglycinyl)]-2′-[N′-(N-benzyloxycarbonyl-L-leucinyl)]carbohydrazide;

(3RS)-1-(N-benzyloxycarbonyl-L-leucinyl)-3-[N-(4-phenoxybenzoyl)amino]pyrrolidin-4-one;

(1S)-N-[2-[1-(N-benzyloxycarbonylamino)-3-methylbutyl]thiazol-4-ylcarbonyl]-N′-[N-(2-pyridinylmethoxycarbonyl)-L-leucinyl]hydrazide;

1-(N-benzyloxycarbonyl-L-leucinylamino)-3-(2-benzyloxyphenylsulfonyl)amino-propan-2-one;

N-[2-(2-benzyloxyphenyl)thiazol-4-ylcarbonyl]-N′-[N-(3-pyridinylmethoxycarbonyl)-L-leucinyl]hydrazide;

(1S)-N-[2-[1-(N-benzyloxycarbonylamino)-3-methylbutyl]thiazol4-ylcarbonyl]-N′-[N-(3-pyridinylmethoxycarbonyl)-L-leucinyl]bydrazide;

1-[N-(4-morpholinocarbamoyl)-L-leucinylamino]-3-(4-phenoxyphenylsulfonyl)amino-propan-2-one;

N-[2-(1-naphthyl)thiazol-4-ylcarbonyl]-N′-[N-(4-pyridinylmethoxycarbonyl)-L-leucinyl]hydrazide;

N-[2-(2-benzyloxyphenyl)thiazol-4-ylcarbonyl]-N′-(N-pyrazinecarbonyl-L-leucinyl)hydrazide;

N-[N-(1-benzyl-5-methylimidazol-4-ylcarbonyl)-L-leucinyl]-N′-[2-(1-naphthyl)thiazol-4-ylcarbonyl]hydrazide;

(3RS)-3-[N-(3-benzyloxybenzoyl)-L-leucinylamino]tetrabydrofuran-4-one;

N-[2-[N-cyclopropyl-N-(2-methylpropyl)amino]thiazol-4-ylcarbonyl]-N′-[N-(5-methyl-2-phenyloxazol-4-ylcarbonyl)-L-leucinyl]hydrazide;

1-[3-(2-pyridinyl)phenylacetylamino]-3-[N-(2-thiophenecarbonyl)-L-leucinylamino]propan-2-one;

(3S)-3-[N-(benzothiazol-6-ylcarbonyl)-L-leucinylamino]- 1-[3-(2-pyridinyl)phenylacetylamino]butan-2-one;

N-[2-[N-cyclopropyl-N-(2-methylpropyI)amino]thiazol-4-ylcarbonyl]-N′-tN-(7-methoxybenzofuran-2-ylcarbonyl)- L-b-cyclopropylalanyl]hydrazide;

1-[N-(benzoxazol-5-ylcarbonyl)-L-leucinylamino]-3-[3-(2-pyridinyl)phenylacetylamino]propan-2-one;

1 -[N-[4-[2-(N,N-dimethylamino)ethoxy]benzoyl]-L-leucinylamino]-3-[3-(2-pyridinyl)phenylacetylaminolpropan-2-one; and

N-[2-[N-cyclopropyl-N-(2-methylpropyl)aminolthiazol-4-ylcarbonyl]-N′-[N-[5-[2-(N,N-dimethylamino)ethoxy]benzofuran-2-ylcarbonyl]-L-b-cyclopropylalanyl]hydrazide.

In particular, these compounds are used in the present method to treat diseases, in particular parasitic diseases, by inhibiting cysteine protease of the papain superfamily. Most particularly, the present invention provides a method of treating malaria by the inhibition of falcipain with such compounds.

### DETAILED DESCRIPTION OF THE INVENTION

The present invention provides a method for treating diseases, particularly parasitic diseases, which may be therapeutically modified by altering the activity of cysteine proteases by administering to a patient in need thereof, particularly an animal, more particularly a mammal, most particularly a human being, one or more of the following compounds:

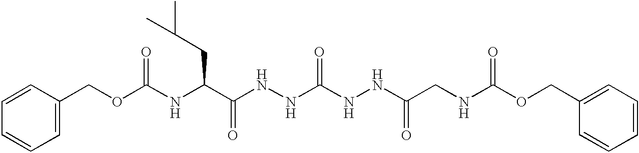

(R)2-[N-(N-benzyloxycarbonylglycinyl)]-2-[N′-(N-benzyloxycarbonyl-L-leucinyl)]carbohydrazide;

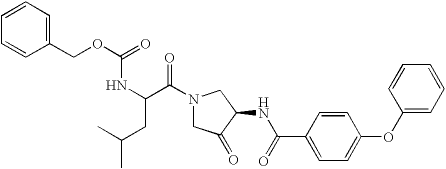

(3RS)-1-(N-benzyloxycarbonyl-L-leucinyl)-3-[N-(4-phenoxybenzoyl)aminolpyrrolidin-4-one;

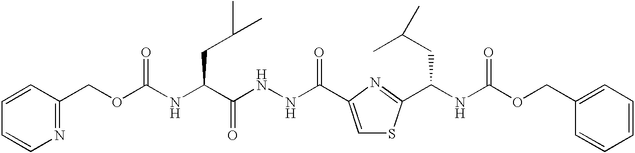

(1S)-N-[2-[l-(N-benzyloxycarbonylamino)-3-methylbutyl]thiazol-4-ylcarbonyl}-N′-[N-(2-pyridiny]methoxycarbonyl)-L-leucinyl]hydrazide;

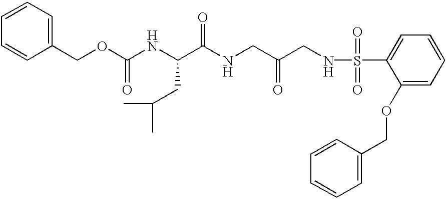

1-(N-benzyloxycarbonyl-L-leucinylamino)-3-(2-benzyloxyphenylsulfonyl)amino-propan-2-one;

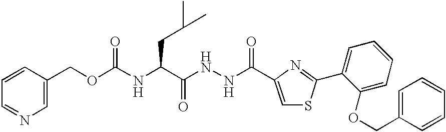

N-[2-(2-benzyloxyphenyl)tbiazol-4-ylcarbonyl]-N′-[N-(3-pyridinylmethoxycarbonyl)-L-leucinyl]hydrazide;

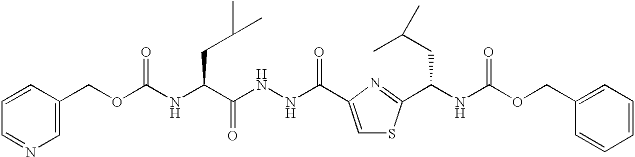

(1S)-N-[2-[1-(N-benzyloxycarbonylamino)-3-methylbutyl]thiazol-4-ylcarbonyl]-N′-[N-(3-pyridinylmethoxycarbonyl)-L-leucinyl]hydrazide;

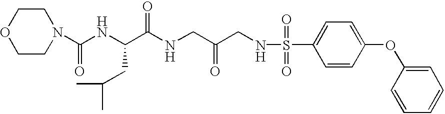

1-[N-(4-morpholinocarbamoyl)-L-leucinylaminol-3-(4-phenoxyphenylsulfonyl)amino-propan-2-one;

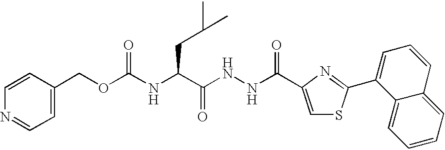

N-2-(1-naphthyl)thiazol-4-ylcarbonyl]-N-l[N-(4-pyridinylmethoxycarbonyl)-L-leucinyl]hydrazide;

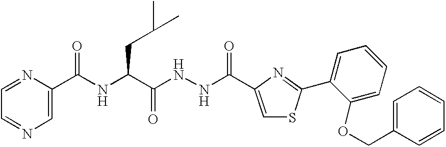

N-[2-(2-benzyloxyphenyl)thiazol-4-ylcarbonyl]-N7-(N-pyrazinecarbonyl-L-leucinyl)hydrazide;

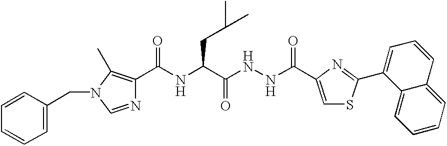

N-[N-(1-benzyl-5-methylimidazol-4-ylcarbonyl)-L-leucinyl]-N′-[2-(1-naphthyl)thiazol-4-ylcarbonyl]hydrazide;

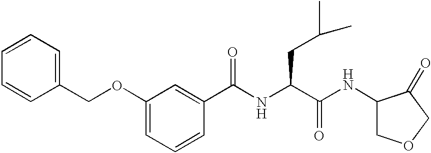

(3RS)-3-[N-(3-benzyloxybenzolyl-L-leucinylamino]tetraydrofuran-4-one;

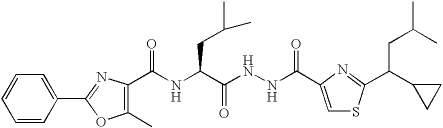

N-[N-(l1-benzyl-5-methylimidazol-4-ylcarbonyl)-L-leucinyl]-N′-[2-(1-naphthyl)thiazol4-ylcarbonyl]hydrazide;

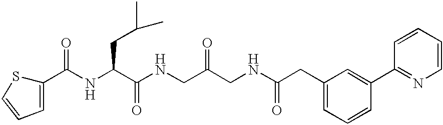

(1-[3-(2-pyrldinyl)phenylacetylamino]-3-[N-(2-thiophenecarbonyl)-L-leucinylamino]propan-2-one;

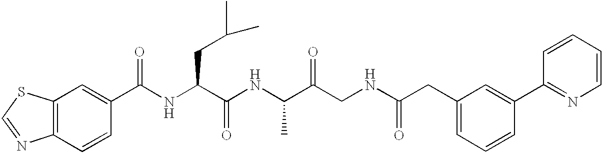

(3S)-3-[N-(benzothiazol-6-ylcarbonyl)-L-teucinylamino]-1-[3-(2-pyridinyl)phenylacetylamino]butan-2-one;

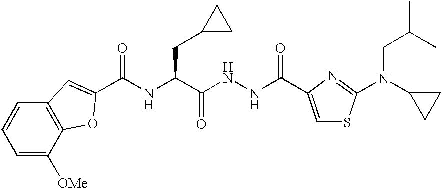

N-[2-[N-cyclopropyl-N-(2-methylpropyl)aminolthiazol-4-ylcarbonyl)-N′-[N-(7-methoxybenzofuran-2-ylcarbonyl)-L-b-cyclopropylalanyl]hydrazide;

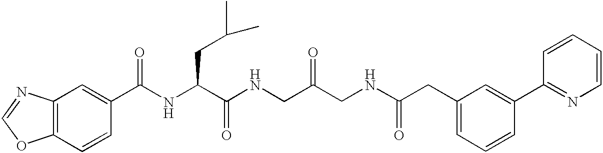

1-[N-(benzoxazol-5-ylcarbonyl)-L-leucinylamino]-3-[3-(2-pyridinyl)phenylacetylaminolpropan-2-one;

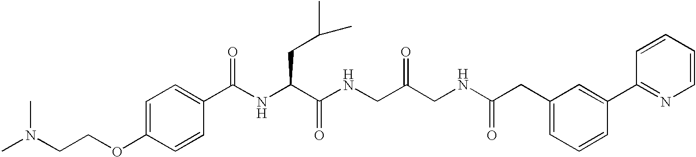

1-[N-[4-[2-(N,N-dimethylamino)ethoxylbenzoyl]-L-leucinylamino]-3-[3-(2-pyridinyl)phenylacetylamino]propan-2-one; and

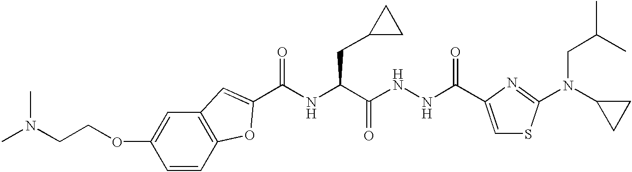

N-[2-[N-cyclopropyl-N-(2-methylpropyl)amiiro]thiazol-4-ylcarbonyl]-N′-[N-[5-[2-(N,N-dimethylamino)ethoxy]benzofuran-2-ylcarbonyl]-L-b-cyclopropylalanyl]hydrazide.

In particular, the present method provides treatment of diseases, particularly parasitic diseases, by inhibiting cysteine proteases of the papain superfamily by administering to a patient in need thereof, particularly an animal, more particularly a mammal, most particularly a human being, one or more of the above-listed compounds.

Parasites known to utilize cysteine proteases in their life cycle include Trypanosoma cruzi, Trypanosoma Brucei [trypanosomiasis (African sleeping sickness, Chagas disease)], Leishmania mexicana, Leishmantia pifanoi, Leishmania major (leishmaniasis), Schistosoma mansoni (schistosomiasis), Onchocerca volvulus [onchocerciasis (river blindness)] Brugia pahangi, Entamoeba histolytica, Giardia lambia, the helminths, Haemonchus contortus and Fasciola hepatica, as well as helminths of the genera Spirometra, Trichinella, Necator and Ascaris, and protozoa of the genera Cryptosporidium, Eimeria, Toxoplasma and Naegleria. The present method provides treatment of diseases caused by infection by these parasites by inhibiting cysteine proteases of the papain superfamily by administering to a patient in need thereof, particularly an animal, more particularly a mammal, most particularly a human being, one or more of the above-listed compounds.

Most particularly, the present invention provides a method of treating malaria, caused by infection with Plasmodium falciparum, by the inhibition of falcipain by administering a patient in need thereof, particularly an animal, more particularly a mammal, most particularly a human being, one or more of the above-listed compounds.

The present method may be practiced by administering the above-listed compounds alone or in combination with other therapeutically effective compounds.

Certain radical groups are abbreviated herein. t-Bu refers to the tertiary butyl radical, Boc refers to the t-butyloxycarbonyl radical, Fmoc refers to the fluorenylmethoxycarbonyl radical, Ph refers to the phenyl radical, Cbz refers to the benzyloxycarbonyl radical.

The present invention includes all esters, hydrates, solvates, complexes and prodrugs of the above-listed compounds useful in the inventive method. Prodrugs are any covalently bonded compounds which release the active parent drug in vivo. If a chiral center or another form of an isomeric center is present in a compound of the present invention, all forms of such isomer or isomers, including enantiomers and diastereomers, are intended to be covered herein. Inventive compounds containing a chiral center may be used as a racemic mixture, an enantiomerically enriched mixture, or the racemic mixture may be separated using well-known techniques and an individual enantiomer may be used alone. In cases in which compounds have unsaturated carbon-carbon double bonds, both the cis (Z) and trans (E) isomers are within the scope of this invention. In cases wherein compounds may exist in tautomeric forms, such as keto-enol tautomers. each tautomeric form is contemplated as being included within this invention whether existing in equilibrium or predominantly in one form.

Synthesis Methods

The following synthesis protocols refer to intermediate compounds and final products identified in the specification and in the synthesis schemes. The preparation of the compounds of the present invention are described in detail using the following examples, but the chemical reactions described are disclosed in terms of their general applicability to the preparation of the cysteine protease inhibiting compounds of the invention. Occasionally, the reaction may not be applicable as described to each compound included within the disclosed scope of the invention. The compounds for which this occurs will be readily recognized by those skilled in the art. In all such cases, either the reactions can be successfully performed by conventional modifications known to those skilled in the art, that is, by appropriate protection of interfering groups, by changing to other conventional reagents, or by routine modifications of reaction conditions. Alternatively, other reactions disclosed herein or otherwise conventional will be applicable to the preparation of the corresponding compounds of the invention. In all preparative methods all starting materials are known or readily preparable from known starting materials; all temperatures are set forth in degrees Celsius; and, unless otherwise indicated, all parts and percentages are by weight.

Reagents were purchased from commercial suppliers such as Aldrich Chemical Company, TCI, Sigma, Lancaster Synthesis, Bionet, Fluka, Maybridge or Bachem, and were used without further purification unless otherwise indicated. All solvents were purified by using standard methods readily known to those skilled in the art unless otherwise indicated. Starting materials are commercially available or were prepared by routine methods as can be found in standard reference books, such as the Compendium of Organic Synthetic Methods, Vol. I-IV (published by Wiley-Interscience).

Coupling methods to form amide bonds herein are generally well-known to the art. The methods of peptide synthesis generally set forth by Bodansky et al., THE PRACTICE OF PEPTIDE SYNTHESIS, Springer-Verlag, Berlin, 1984; E. Gross and J. Meienhofer, THE PETIDES, Vol. 1, 1-284 (1979); and J. M. Stewart and J. D. Young, SOLID PHASE PEPTIDE SYNTHESIS, 2d Ed., Pierce Chemical Co., Rockford, Ill., 1984, are generally illustrative of the technique and are incorporated herein by reference.

Synthetic methods to prepare the compounds of this invention frequently employ protective groups to mask a reactive functionality or minimize unwanted side reactions. Such protective groups are described generally in Green, T. W, PROTECTIVE GROUPS IN ORGANIC SYNTHESIS, John Wiley & Sons, New York (1981). The term “amino protecting groups” generally refers to the Boc, acetyl, benzoyl, Fmoc and Cbz groups and derivatives thereof as known to the art. Methods for protection and deprotection, and replacement of an amino protecting group with another moiety are well known.

Acid addition salts of the above-listed compounds useful in the inventive method are prepared in a standard manner in a suitable solvent from the parent compound and an acid, such as hydrochloric, hydrobromic, hydrofluoric, sulfuric, phosphoric, acetic, trifluoroacetic, maleic, succinic or methanesulfonic acid.

Some of the compounds described herein contain one or more centers of asymmetry and may thus give rise to enantiomers, diastereoisomers, and other stereoisomeric forms. The present invention is meant to include all such possible stereoisomers as well as their racemic and optically pure forms. Optically active (R) and (S) isomers may be prepared using chiral synthons, chiral reagents, or resolved using conventional techniques. When the compounds described herein contain olefinic double bonds, it is intended to include both E and Z geometric isomers.

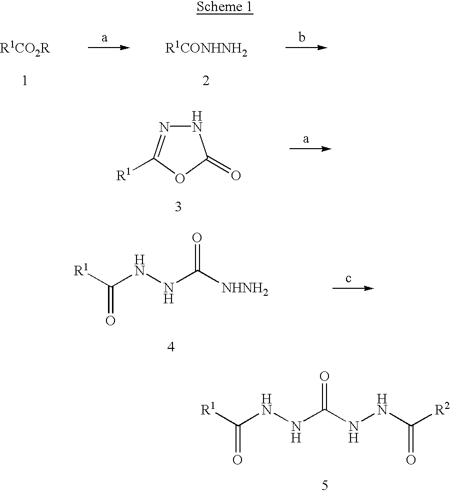

a) H₂NNH₂.H₂O, MeOH; b) Cl₂CO, PhMe; c) R²CO₂H, EDC.HCl, 1-HOBT, DMF.

Treatment of 1-Scheme 1 with hydrazine hydrate in a protic solvent (such as methanol or ethanol) provided 2-Scheme 1, which was treated with phosgene in toluene to afford 3-Scheme 1. This material was treated with hydrazine hydrate in a protic solvent (such as methanol or ethanol) to provide 4-Scheme 1. Treatment of 4-Scheme 1 with a carboxylic acid (such as N-benzyloxycarbonylglycine) and a peptide coupling reagent (such as EDC.HCl/1-HOBT) in an aprotic solvent (such as DMF) provided 5-Scheme 1.

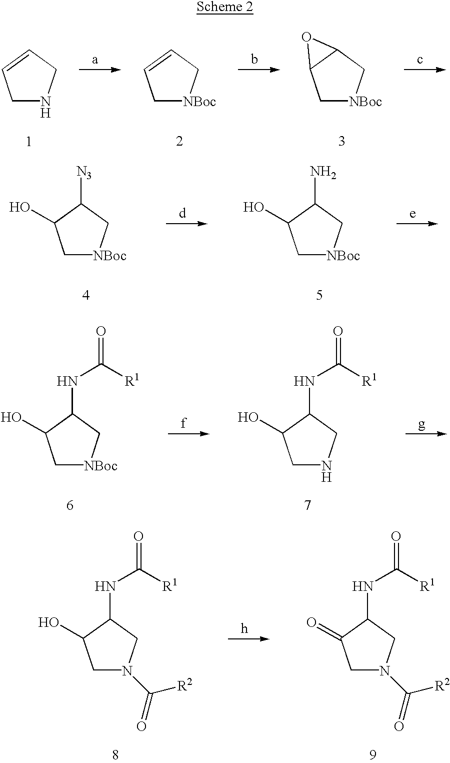

a) Boc₂O, CH₂Cl₂; b) m-CPBA, CH₂Cl₂; c) NaN₃, NH₄Cl, MeOH/H₂O; d) H₂, 10% Pd/C, MeOH; e) R¹CO₂H, EDC.HCl, l-HOBT, DMF; f) HCl, EtOAc; g) R²CO₂H, EDC-HCl, 1-HOBT, DMF; h) Jones reagent, acetone.

Treatment of 1-Scheme 2 with di-tert-butyl dicarbonate in methylene chloride provided 2-Scheme 2, which was treated with m-chloroperbenzoic acid in methylene chloride to afford 3-Scheme 2. Treatment of this material with sodium azide and ammonium chloride in methanol/water gave 4-Scheme 2, which was treated with hydrogen gas in the presence of 10% palladium on carbon in methanol to give 5-Scheme 2. Treatment of this material with a carboxylic acid (such as 4-phenoxybenzoic acid) and a peptide coupling reagent ( such as EDC-HCl/1-HOBT) in an aprotic solvent (such as DMF) provided 6-Scheme 2, which was treated with HCl gas in ethyl acetate to provide 7-Scheme 2. Treatment of 7-Scheme 2 with a carboxylic acid (such as N-benzyloxycarbonyl-L-leucine) and a peptide coupling reagent ( such as EDC*HCll1-HOBT) in an aprotic solvent (such as DMF) provided 8-Scheme 2, which was treated with Jones reagent in acetone to provide 9-Scheme 2.

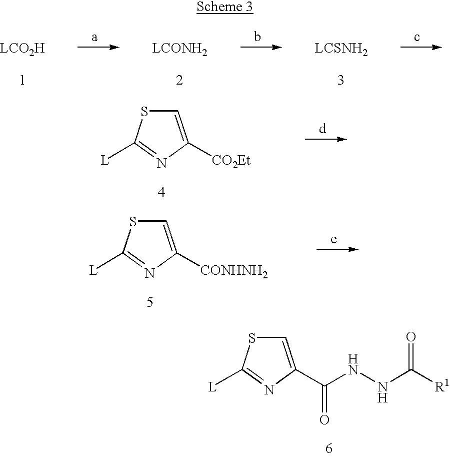

a) i-BuOCOCI, NMM, NH3, THF; b) Lawesson's reagent, THF; c) i. EtO2CCOCH₂Br; ii. TFAA, Py, CH₂Cl₂; d) H₂NNH₂.H₂O, EtOH; e) R¹CO₂H, EDC.HCl, 1-HOBT, DMF.

1-Scheme 3 was converted to 2-Scheme 3 by treatment with isobutyl chloroformate, N-methylmorpholine and ammonia in THF. 2-Scheme 3 was treated with Lawesson's reagent in THF to provide the thioamide 3-Scheme 3. This material was converted to the thiazole by condensation with an a-ketoester followed by treatment with trifluoroacetic anhydride and pyridine in methylene chloride to afford 4-Scheme 3 which was converted to 5-Scheme 3 by treatment with hydrazine monohydrate. Treatment of 5-Scheme 3 with a carboxylic acid (such as N-(2-pyridinylmethoxycarbonyl)-L-leucine or N-(3-pyridinylmethoxycarbonyl)-L-leucine) and a peptide coupling reagent (such as EDC.HCl/1-HOBT) in an aprotic solvent (such as DMF) provided 6-Scheme 3.

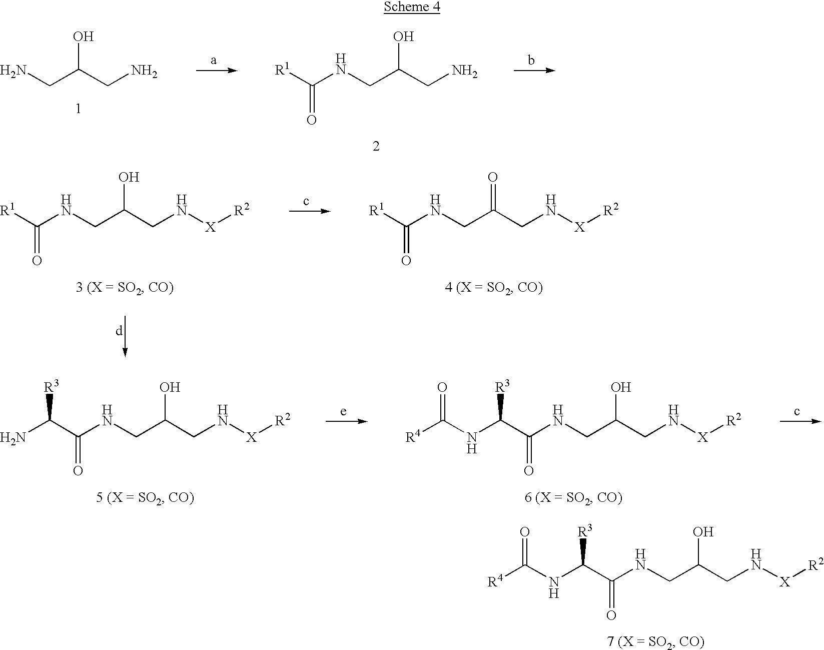

a) RIC0₂H, EDC.MeI, 1-HOBT, DMF; b) R²SO₂Cl, N-methylmorpholine, DMF or R²CO₂H, EDC.MeI, 1-HOBT, DMF; c) Jones reagent, acetone or Dess-Martin reagent, CH₂Cl₂; d) for R¹=N-benzyloxycarbonyl-amino acid, H₂, 10% Pd/C, EtOH; for R¹=tert-butoxycarbonyl-amino acid, TFA, CH₂Cl₂; e) R⁴CO₂H, EDC-MeI, 1-HOBT, DMF.

Treatment of 1-Scheme 4 with a carboxylic acid (such as N-benzyloxycarbonyl-L-leucine or N-tert-butoxycarbonyl-L-leucine) and a peptide coupling reagent (such as EDC.HCl/1-HOBT, EDC-MeI/1-HOBT or HBTU) in an aprotic solvent (such as DMF) provided 2-Scheme 4, which was treated with a sulfonyl chloride (such as 2-benzyloxyphenylsulfonyl chloride or 4-phenoxyphenylsulfonyl chloride) and N-methylmorpholine in an aprotic solvent (such as DMF) to give 3-Scheme 4. Alternatively, 2-Scheme 4 was treated with a carboxylic acid (such as 3-(2-pyridinyl)phenylacetic acid) and a peptide coupling reagent (such as EDC.HCl/1-HOBT, EDC.Mel/1-HOBT or HBTU) in an aprotic solvent (such as DMF) to provide 3-Scheme 4. Treatment of 3-Scheme 4 with Jones reagent in acetone or Dess-Martin reagent in methylene chloride then gave 4-Scheme 4. When RICO was a N-benzyloxycarbonyl-amino acid, treatment of 3-Scheme 4 with hydrogen gas in the presence of 10% palladium on carbon in ethanol provided 5-Scheme 4. Alternatively, when RICO was a N-tert-butoxycarbonyl-amino acid, treatment of 3-Scheme 4 with trifluoroacetic acid in dichloromethane provided 5-Scheme 4. Treatment of 5-Scheme 4 with a carbamoyl chloride (such as 4-morpholine carbonyl chloride) and a tertiary amine base (such as N-methylmorpholine) in an aprotic solvent (such as DMF) provided 6-Scheme 4. Alternatively, 5-Scheme 4 was treated with a carboxylic acid (such as thiophene-2-carboxylic acid, benzoxazole-5-carboxylic acid or 4-[2-(N,N-dimethylamino)ethyoxy]benzoic acid) and a peptide coupling reagent (such as EDC.HCl/1-HOBT, EDCoMeI/1-HOBT or HBTU) in an aprotic solvent (such as DMF) to provide 6-Scheme 4. Treatment of 6-Scheme 4 with Dess-Martin reagent in methylene chloride provided 7-Scheme 4.

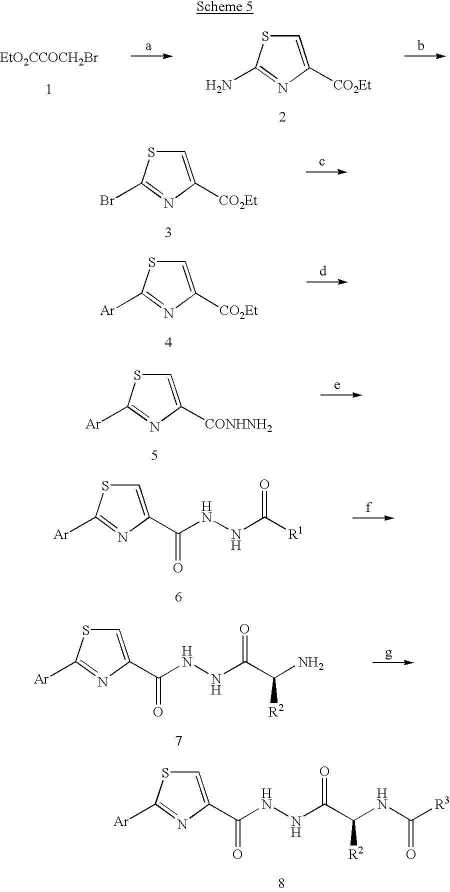

a) Thiourea, EtOH; b) i.NaNO₂, 16% aqueous HBR; ii. CuBr, 16% aqueous HBr; iii. HBr (cat.), EtOH; c) ArB(OH)₂Pd(PPh₃)₄, CsF, DME; d) H₂NNH₂. H₂O, EtOH; e) R¹CO₂H, EDC.HCl, 1-HOBT, DMF; f) for R¹CO₂H=N-tert-butoxycarbonyl-amino acid; TFA, CH₂Cl₂; g) R³CO₂H, EDC.HCl, 1-HOBT, DMF.

Ethyl bromopyruvate (1-Scheme 5 was treated with thiourea in refluxing ethanol to provide 2-Scheme 5, which was treated successively with sodium nitrite and copper (I) brom ide in 16% aqueous HBr, and the product was heated in ethanol with a catalytic amount of HBr to give 3-Scheme 5. Treatment of this material with an arylboronic acid (such as 2-benzyloxyphenylboronic acid or 1-naphthylboronic acid), tetrakis(triphenylphosphine)palladiumn(O) and cesium fluoride in refluxing DME provided 4-Scheme 5. Treatment of 4-Scheme 5 with hydrazine hydrate in ethanol provided 5-Scheme 5, which was treated with a carboxylic acid (such as N-tert-butoxycarbonyl-L-leucine, N-(3-pyridinylmethoxycarbonyn-L-leucine or N-(4-pyridinelmethoxycarbonyl)-L-leucine) and a peptide coupling reagent (such as EDCmHCil-HOBit in an aprotic solvent (such as DMF) to provide 6-Scheme 5. Where R¹CO was an N-tert-butoxycarbonyl-amino acid, 6-Scheme 5 was treated with trifluoroacetic acid in dichloromethane to provide 7-Scheme 5, which was treated with a carboxylic acid (such as pyrazinecarboxylic acid or 1-benzyl-5-methylimidazole-4carboxylic acid) and a peptide coupling reagent (such as EDC.HCl/1-HOBT) in an aprotic solvent (such as DMF) to provide 8-Scheme 5.

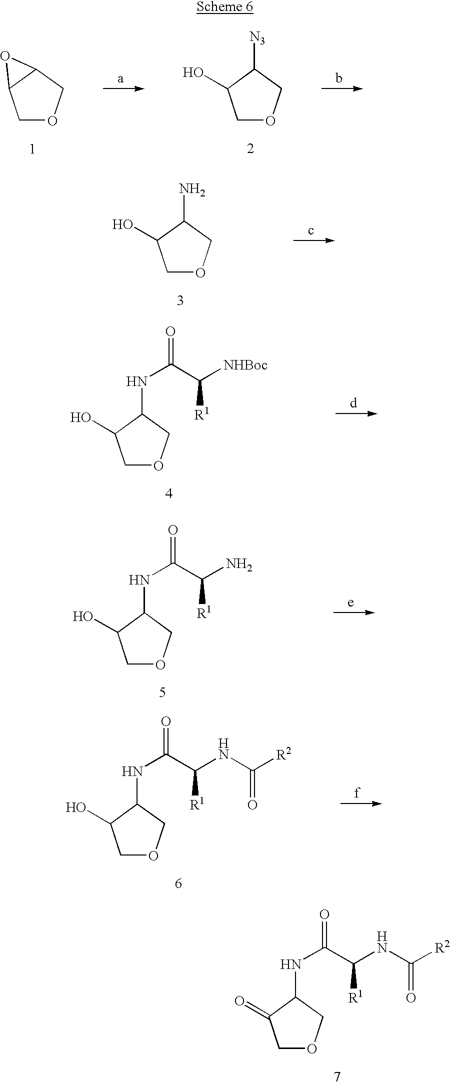

a) NaN₃, NR₄Cl, MeOH/water; b) H₂, 10% Pd/C, EtOH; c) BocNHCH(R¹)CO₂H, Me₃COCl, N,N-diisopropyletliylamine, CH₂Cl₂; d) TFA, CH₂Cl₂; e) R²CO₂H, (EtO)₂POCN, Et₃N, CH₂Cl₂; f) Dess-Martin reagent, CH₂Cl₂.

Treatment of 1-Scheme 6 with sodium azide and ammonium chloride in methanol/water gave 2-Scheme 6, which was treated with hydrogen gas in the presence of 10% palladium on carbon in ethanol to give 3-Scheme 6. Treatment of 3-Scheme 6 with a N-tert-butoxycarbonyl amino acid, pivaloyl chloride and a tertiary amine base (such as N,N-diisopropylamine) in an aprotic solvent (such as dichloromethane) provided 4-Scheme 6, which was treated with triflouroacetic acid in dichloromethane to provide 5-Scheme 6. Treatment of 5-Scheme 6 with a carboxylic acid (such as 3-benzyloxybenzoic acid) and a peptide coupling reagent (such as EDC.HCl/1-HOBT, EDC-MeI/1-HOBT, HBTU or diethyl cyanophosphonate) in an aprotic solvent (such as DMF or dichloromethane) provided 6-Scheme 6, which was treated with Dess-Martin reagent in dichloromethane to provide 7-Scheme 6.

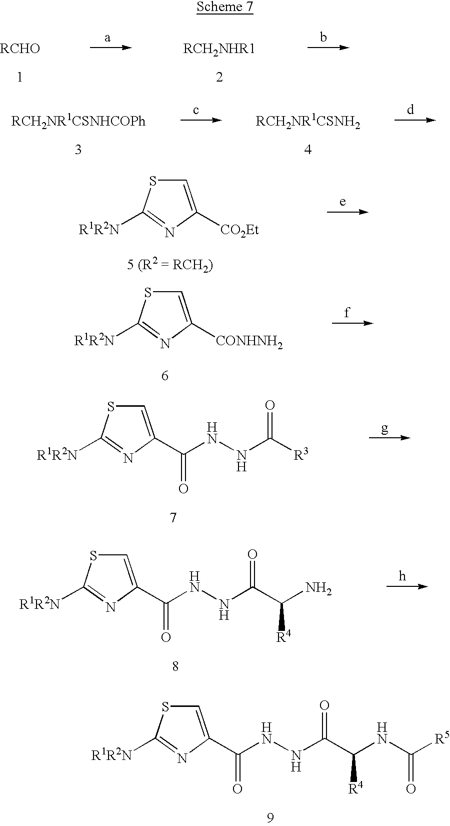

a) i. R¹NH₂, CH₂Cl₂; ii. Na(OAc)₃BH; b) PhCONCS, CHCl₃; c) K₂CO₃, MeOH, H₂O; d) EtO₂CCOCH₂Br, EtOH; e) H₂NNH₂.H₂O, EtOH; f) R³CO₂H, EDC.HCl, 1-HOBT, DMF; g) for R³C0₂H =N-tert-butoxycarbonyl-amino acid: TFA, CH₂Cl₂; h) R⁵CO₂H, EDC.HCl, 1-HOBT, DMF.

An aldehyde (1-Scheme 7) was treated with a primary amnine (such as cyclopropylamine) in an aprotic solvent (such as methylene chloride), followed by treatment with a reducing agent (such as sodium triacetoxyborohydride), to provide 2-Scheme 7, which was treated with benzoyl isothiocyanate in chloroform to afford 3-Scheme 7. Treatment of 3-Scheme 7 with potassium carbonate in methanol/water provided 4-Scheme 7, which was treated with ethyl bromopyruvate in refluxing ethanol to give 5-Scheme 7, which was subsequently treated with hydrazine hydrate in ethanol to give 6-Scheme 7. Treatment of 6-Scheme 7 with a carboxylic acid (such as N-tert-butoxycarbonyl-L-leucine) and a peptide coupling reagent (such as EDC-HCl/1-HOBT) in an aprotic solvent (such as DMF) gave 7-Scheme 7. Where R³CO was an N-tert-butoxycarbonyl-amino acid, 7-Scheme 7 was treated with trifluoroacetic acid in dichloromethane to provide 8-Scheme 7, which was treated with a carboxylic acid (such as 5-methyl-2-phenyloxazole-4-carboxylic acid, 7-methoxybenzofuran-2-carboxylic acid or 5-[2-(N,N-dimethylamino)ethoxy]benzofuran-2-carboxylic acid) and a peptide coupling reagent (such as EDC-HCl/1-HOBT) in an aprotic solvent (such as DMF) to provide 9-Scheme 7.

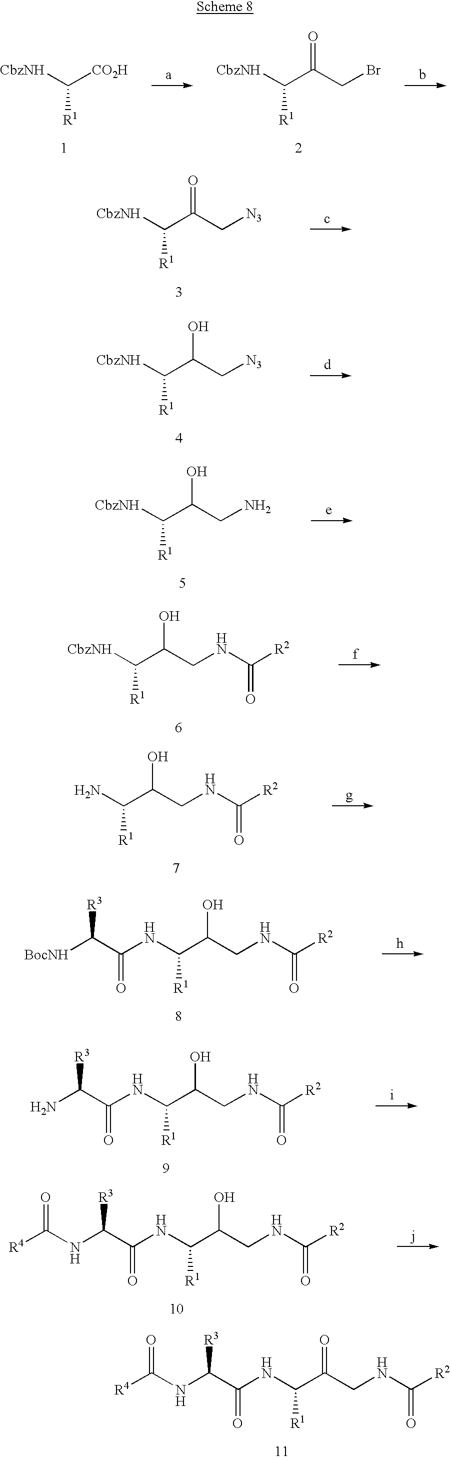

a) i. Isobutyl chloroformate, N-methylmorpholine, THF; ii. CH₂N₂, Et₂O; iii. 30% HBr/HOAc; b) NaN₃, KF, DMF; c) NaBH₄, MeOH; d) 1,3-propanedithiol, Et₃N, MeOH; e) R²CO₂H, EDC.MeI, 1-HOBT, DMF; f) H₂, 10% Pd/C, EtOH; g) BocNHCH(R³)CO₂H, HBTU, DMF; h) HCl, dioxane, CH₂CI₂; i) R⁴CO₂H, HBTU, DMF; j) Dess-Martin reagent, CH₂Cl₂.

Sequential treatment of 1-Scheme 8 with isobutyl chloroformate and N-methylmorpholine in THF, diazomethane in ether, and 30% HBr in acetic acid provided 2-Scheme 8, which was treated with sodium azide and potassium flouride in DMF to provide 3-Scheme 8. Treatment of 3-Scheme 8 with sodium borohydride in methanol provided 4-Scheme 8, which was treated with 1,3-propanedithiol and triethylamine in methanol to afford 5-Scheme 8. Treatment of 5-Scheme 8 with a carboxylic acid (such as 3-(2-pyridinyl)phenylacetic acid) and a peptide coupling reagent (such as EDC-HCl/1-HOBT, EDC.MeI/1-HOBT or HBTU) in an aprotic solvent (such as DMF) provided 6-Scheme 8, which was treated with hydrogen gas in the presence of 10% palladium on carbon in ethanol to give 7-Scheme 8. Treatment of 7-Scheme 8 with a N-tert-butoxycarbonyl amino acid (such as N-tert-butoxycarbonyl-L-leucine) and a peptide coupling reagent (such as EDC.HCl/1-HOBT, EDC.MeI/1-HOBT or HBTU) in an aprotic solvent (such as DMF) provided 8-Scheme 8, which was treated with HCl in dioxane/dichloromethane to provide 9-Scheme 8. Treatment of 9-Scheme 8 with a carboxylic acid (such as benzothiazole-6-carboxylic acid) and a peptide coupling reagent (such as EDC.HCl/1-HOBT, EDC.MeI/1-HOBT or HBTU) in an aprotic solvent (such as DMF) provided 10-Scheme 8, which was treated with Dess-Martin reagent in dichloromethane to provide 11-Scheme 8.

This invention also provides pharmaceutical compositions which comprise one or more of the following compounds:

2-[N-(N-benzyloxycarbonylglycinyl)]-2′-[N′-(N-benzyloxycarbonyl-L-leucinyl)]carbohydrazide;

(3RS)-1-(N-benzyloxycarbonyl-L-leucinyl)-3-[N-(4-phenoxybenzoyl)aminolpyrrolidin-4one;

(1S)-N-[2-[1-(N-benzyloxycarbonylamino)-3-methylbutyl]thiazol-4-ylcarbonyl[-N′-[N-(2-pyridinylmethoxycarbonyl)-L-leucinyllhydrazide;

1-(N-benzyloxycarbonyl-L-leucinylamino)-3-(2-benzyloxyphenyisulfonyl)aino-propan-2-one;

N-[2-(2-benzyloxyphenyl)thiazol-4-ylcarbonyl]-N′-[N-(3-pyridinylmethoxycarbonyl)-L-leucinyl]hydrazide;

(1S)-N-[2-[1-(N-benzyloxycarbonylamino)-3-methylbutyllthiazoi-4-ylcarbonyl]-N′-[N-(3-pyridinylmethoxycarbonyl)-L-leucinyl]hydrazide;

1-[N-(4-morpholinocarbamoyl)-L-leucinylamino]-3-(4-phenoxyphenylsulfonyl)amino-propan-2-one;

N-[2-(1-naphthyl)thiazol-4-ylcarbonyl]-N′-[N-(4-pyridinylmethoxycarbonyl)-L-leucinyl]hydrazide;

N-[2-(2-benzyloxyphenylthiazol-4-ylcarbonyl]-N′-(N-pyrazinecarbonyl-L-leucinyl)hydrazide;

N-[N-(1-benzyl-5-methylimidazol-4-ylcarbonyl)-L-leucinyl]-N′-[2-(1-naphthyl)thiazol-4-ylcarbonyl]hydrazide;

(3RS)-3-[N-(3-benzyloxybenzoyl)-L-leucinylaminojtetrahydrofuran-4-one;

N-[2-[N-cyclopropyl-N-(2-methylpropyl)aminolthiazol-4-ylcarbonyl]-N′-[N-(5-methyl-2-phenyloxazol-4-ylcarbonyl)-L-leucinyl]hydrazide;

1-[3-(2-pyridinyl)phenylacetylamino]-3-[N-(2-thiophenecarbonyl)-L-leucinylaminolpropan-2-one;

(3S)-3-[N-(benzothiazol-6-ylcarbonyl)-L-leucinylaminol-1-[3-(2-pyridinyl)phenylacetylaminolbutan-2-one;

N-[2-[N-cyclopropyl-N-(2-methylpropyl)aminolthiazol-4-ylcarbonyll-N′-[N-(7-methoxybenzofuran-2-ylcarbonyl)- L-b-cyclopropylalanyl]hydrazide;

1-[N-(benzoxazol-5-ylcarbonyl)-L-leucinylamino]-3-[3-(2-pyridinyl)phenylacetylamino]propan-2-one;

1-[N-[4-[2-(N,N-dimethylamino)ethoxy]benzoyl]-L-teucinylamino]-3-[3-(2-pyridinyl)phenylacetylaminolpropan-2-one; and

N-[2-[N-cyclopropyl-N-(2-methylpropyl)amino]thiazol4-ylcarbonyl]-N′-[N-[5-[2-(N,N-dimethylamino)ethoxy]benzofuran-2-ylcarbonyl]- L-b-cyclopropylalanyl]hydrazide and a pharmaceutically acceptable carrier, diluent or excipient. Accordingly, the above-listed compounds may be used in the manufacture of a medicament. Pharmaceutical compositions of the above-listed compounds prepared as hereinbefore described may be - - formulated as solutions or lyophilized powders for parenteral administration. Powders may be reconstituted by addition of a suitable diluent or other pharmaceutically acceptable carrier prior to use. The liquid formulation may be a buffered, isotonic, aqueous solution. Examples of suitable diluents are normal isotonic saline solution, standard 5% dextrose in water, or buffered sodium or ammonium acetate solution. Such formulation is especially suitable for parenteral administration, but may also be used for oral administration or contained in a metered dose inhaler or nebulizer for insufflation. It may be desirable to add excipients such as polyvinylpyrrolidone, gelatin, hydroxy cellulose. acacia, polyethylene glycol, mannitol, sodium chloride, or sodium citrate.

Alternately, these compounds may be encapsulated, tableted, or prepared in an emulsion or syrup for oral administration. Pharmaceutically acceptable solid or liquid carriers may be added to enhance or stabilize the composition, or to facilitate preparation of the composition. Solid carriers include starch, lactose, calcium sulfate dihydrate, terra alba, magnesium stearate or stearic acid, talc, pectin, acacia, agar or gelatin. Liquid carriers include syrup, peanut oil, olive oil, saline and water. The carrier may also include a sustained release material such as glyceryl monostearate or glyceryl distearate, alone or with a wax. The amount of solid carrier varies but, preferably, will be between about 20 mg to about 1 g per dosage unit. The pharmaceutical preparations are made following the conventional techniques of pharmacy involving milling, mixing, granulating, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly or filled into a soft gelatin capsule.

For rectal administration, the compounds of this invention may also be combined with excipients such as cocoa butter, glycerin, gelatin or polyethylene glycols and molded into a suppository.

In accordance with this invention, an effective amount one or more of the above-listed compounds is administered to inhibit the protease implicated with a particular condition or disease. Of course, this dosage amount will further be modified according to the type of administration of the compound. For example, for acute therapy, parenteral administration of an effective amount of an inventive compound is preferred. An intravenous infusion of the compound in 5% dextrose in water or normal saline, or a similar formulation with suitable excipients, is most effective, although an intramuscular bolus injection is also useful. Typically, the parenteral dose will be about 0.01 to about 100 mg/kg; preferably between 0.1 and 20 mg/kg, in a manner to maintain the concentration of drug in the plasma at a concentration effective to inhibit the protease, e.g. falcipain. The compound is administered one to four times daily at a level to achieve a total daily dose of about 0.4 to about 400 mg/kg/day. The precise amount of an inventive compound which is therapeutically effective, and the route by which such compound is best administered, is readily determined by one of ordinary skill in the art by comparing the blood level of the agent to the concentration required to have a therapeutic effect.

The compound may be administered in the form of a prodrug which, in general, is designed to enhance absorption and is cleaved in vivo to form the active component. Efficacious levels may also be achieved by administration of pharmaceutically active metabolites or bioisosteres of the compound. Prodrugs of compounds of the present invention may be prepared by any suitable method.

The compounds of this invention may also be administered orally to the patient, in a manner such that the concentration of drug is sufficient to inhibit cysteine proteases, especially falcipain, or to achieve any other therapeutic indication as disclosed herein. Typically, a pharmaceutical composition containing the compound is administered at an oral dose of between about 0.1 to about 50 mg/kg in a manner consistent with the condition of the patient. Preferably the oral dose would be about 0.1 to about 50 mglkg given 1-2 times/day.

No unacceptable toxicological effects are expected when compounds of the present invention are administered in accordance with the present invention.

The compounds of this invention may be tested in one of several biological assays to determine the concentration of a compound which is required to have a given pharmacological effect. For example, an assay for determining Plasmodium falciparum cysteine protease catalytic activity and an assay to determine the amount of cysteine protease inhibition by a compound of the present invention are provided.

All assays for the Plasmodiumfalciparum cysteine protease were carried out with trophozoite extracts (Rosenthal, P. J., et al., J. Clin. Invest 1991, 88, 1467-1472). Standard assay conditions for the determination of kinetic constants used the fluorogenic peptide substrate, Cbz-Phe-Arg-AMC (Bachem) and were determined in 100 mM Na acetate at pH 5.5 containing 5 mM cysteine. Stock substrate solutions were prepared at concentrations of 10 mM in DMS0 with 10 uM final substrate concentration in the assays. The final DMSO concentration was 2 % and the final volume was 100 uL. All assays were conducted at ambient temperature. Product progress curves were generated over 20 to 30 minutes following formation of AMC product.

Potential inhibitors were evaluated using the progress curve method. Assays were carried out in the presence of variable concentrations of test compound. Reactions were initiated by addition of enzyme to buffered solutions of inhibitor and substrate. Data analysis was conducted according to one of two procedures depending on the appearance of the progress curves in the presence of inhibitors. For those compounds whose progress curves were linear, apparent inhibition constants (Kiapp) were calculated according to equation (1) (Brandt et al., Biochemistry, 1989, 28, 140):

ν=VₘA/[Kₐ(1+I/K_{i,app})+A] (1)

where ν is the velocity of the reaction with maximal velocity Vₘ, A is the concentration of substrate with Michaelis constant of Kₐ, and l is the concentration of inhibitor.

For those compounds whose progress curves showed downward curvature characteristic of time-dependent inhibition, the data from individual sets was analyzed to give k_{obs} according to equation (2):

[AMC]=ν_{SS}t+(ν_{O}−ν_{SS})[1−exp (−k_{obs}t)]/k_{obs} (2)

where [AMC] is the concentration of product formed over time t, ν_{O} is the initial reaction velocity and v₅₅ is the final steady state rate. Values for kobs were then analyzed as a linear function of inhibitor concentration to generate an apparent second order rate constant (k_{obs} /inhibitor concentration or k_{obs}/[I]) describing the time-dependent inhibition. A complete discussion of this kinetic treatment has been fully described (Morrison et al., Adv. EnzymoL. Relat. Areas Mol. Biol., 1988, 61, 201).

Exemplary inhibition data for the compounds of the present invention collected in accordance with the above-described procedure are listed in Table I below.

TABLE I

| Compound | Kᵢ(nM) |
|---|---|
| 2-[N-(N-benzyloxycarbonylglycinyl)]-2′-[N′-(N- | 9.5 |
| benzyloxycarbonyl-L-leucinyl)]carbohydrazide | |
| (3RS)-1-(N-benzyloxycarbonyl-L-leucinyl)-3-[N-(4- | 15 |
| phenoxybenzoyl)amino]pyrrolidin-4-one | |
| (1S)-N-[2-[1-(N-benzyloxycarbonylamino)-3- | 55 |
| methylbutyl]thiazol-4-ylcarbonyl]-N′-[N-(2- | |
| pyridinylmethoxycarbonyl)-L-leucinyl]hydrazide | |
| 1-(N-benzyloxycarbonyl-L-leucinylamino)-3-(2- | 54 |
| benzyloxyphenylsulfonyl)amino-propan-2-one | |
| N-[2-(2-benzyloxyphenyl)thiazol-4-ylcarbonyl]-N′-[N-(3- | 41 |
| pyridinylmethoxycarbonyl)-L-leucinyl]hydrazide | |
| (1S)-N-[2-[1-(N-benzyloxycarbonylamino)-3- | 75 |
| methylbutyl]thiazol-4-ylcarbonyl]-N′-[N-(3- | |
| pyridinylmethoxycarbonyl)-L-leucinyl]hydrazide | |
| 1-[N-(4-morpholinocarbamoyl)-L-leucinylamino-3-(4- | 130 |
| phenoxyphenylsulfonyl)amino-propan-2-one | |
| N-[2-(1-naphthyl)thiazol-4-ylcarbonyl]-N′-[N-(4- | 18 |
| pyridinylmethoxycarbonyl)-L-leucinyl]hydrazide | |
| N-[2-(2-benzyloxyphenyl)thiazol-4-ylcarbonyl]- | 28 |
| N′-(N-pyrazinecarbonyl-L-leucinyl)hydrazide | |
| N-[N-(1-benzyl-5-methylimidazol-4-ylcarbonyl)-L-leucinyl]-N′- | 35 |
| [2-(1-naphthyl)thiazol-4-ylcarbonyl]hydrazide | |
| (3RS)-3-[N-(3-benzyloxybenzoyl)- | 230 |
| L-leucinylamino)tetrahydrofuran-4-One | |
| N-[2-[N-cyclopropyl-N-(2-methylpropyl)amino]thiazol-4- | 38 |
| ylcarbonyl]-N′-[N-(5-methyl-2-phenyloxazol-4-ylcarbonyl)-L- | |
| leucinyl]hydrazide | |
| 1-[3-(2-pyridinyl)phenylacetylamino]-3-[N-(2- | 94 |
| thiophenecarbonyl)-L-leucinylamino]propan-2-one | |
| (3S)-3-[N-(benzothiazol-6-ylcarbonyl)-L-leucinylamino]-1-[3-(2- | 81 |
| pyridinyl)phenylacetylamino]butan-2-one | |
| N-[2-[N-cyclopropyl-N-(2-methylpropyl)amino]thiazol-4- | 550 |
| ylcarbonyl]-N′-[N-(7-methoxybenzofuran-2-ylcarbonyl)- | |
| L-b-cyclopropylalanyl]hydrazide | |
| 1-[N-(benzoxazol-5-ylcarbonyl-L-leucinylamino]-3-[3-(2- | 47 |
| pyridinyl)phenylacetylamino]propan-2-one | |
| 1-[N-[4-[2-(N,N-dimethylamino)ethoxy]benzoyl]-L- | 77 |
| leucinylamino]-3-[3-(2-pyridinyl)phenylacetylamino]propan-2- | |
| one | |
| N-[2-[N-cyclopropyl-N-(2-methylpropyl)amino]thiazol-4- | 22 |
| ylcarbonyl]-N′-[N-[5-[2-(N,N- | |
| dimethylamino)ethoxy]benzofuran-2-ylcarbonyl]-L-b- | |
| cyclopropylalanyl]hydrazide | |

The data in Table I demonstrate that the compounds of the present invention are efficacious inhibitors of Plasmodium falciparum cysteine protease, and thus, if administered according to the present method, may be therapeutically effective in treating malaria and other parasitic diseases identified herein above in animals, particularly mammals, most particularly human beings.

### EXAMPLES

In the following synthetic examples, unless otherwise indicated, all of the starting materials were obtained from commercial sources. Without further elaboration, it is believed that one skilled in the art can, using the preceding description, utilize the present invention to its fullest extent. These Examples are given to illustrate the invention, not to limit its scope.

Flash column chromatography was performed using silica gel 60 (Merck Art 9385). ¹H NMR (300 MHz) spectra were measured in CDC13 solutions and were determined on a Varian 300 instrument utilizing a Varian UNITYplus300 operating software. Chemical shifts are reported in parts per million (ppm) downfield from tetramethylsilane as the internal standard, and coupling constants are given in Hertz. The following abbreviations are used for spin multiplicity: br=broad, s=singlet, d=doublet, t=triplet, q=quartet, m =multiplet, cm=complex multiplet. Infrared (IR) spectra were recorded on a Perkin-Elmnef 1600 series FFIR spectrometer and are reported in wave numbers (cm-1).

### EXAMPLE 1

Preparation of 2-[N-(N-benzyloxycarbonylglycinyl)1-2′-[N′-(N-benzyloxvcarbonyl-L-leucinyl)lcarbohydrazide

a) N-benzyloxycarbonyl-L-leucine methyl ester

To a stirring solution of L-leucine methyl ester hydrochloride (2.0 g, 11.0 mmol) in 1,4-dioxane (20 mL) was added Na₂CO₃ (12.1 ml, 2M in water) followed by benzylchloroformate (1.96 g, 11.5 mmol). The mixture was stirred at room temperature for 4 hours then partitioned between ethyl acetate and water. The organic layer was washed with brine, dried (MgSO₄), filtered and concentrated to yield the title compound as a colorless oil (3.1 g, 100%). ¹H NMR (400 MHz, CDCl₃) d 7.34 (m, 5H), 5.27 (d, 1H), 5.12 (s, 2H), 4.41 (s, 2H), 3.75 (s, 3H), 1.65 (m, 3H), 0.96 (m, 6H).

b) N-benzyloxycarbonyl-L-leucinylhydrazide

To a stirring solution of the compound of Example 1(a) (3.1 g, 11.0 mmol) in 15 mL of methanol was added hydrazide hydrate (5.9 g, 118 mmol). The solution was stirred at room temperature for 16 hours then concentrated to yield the title compound as an off-white solid (3.1 g, 100%). MS (ESI): 280.2 (M+H)⁺.

c) (1S)-1-benzyloxycarbonylamino-3-methyl-1-(1,3,4-oxadiazol-2-on-5-yl)butane

To a stirring solution of the compound of Example l(b) (3.0 g, 10.8 mmol) in toluene (50 mL) was added phosgene (56 mL, 1.93M in toluene). The solution was heated at reflux for 4 hours then concentrated to yield the title compound as a pale yellow foam (3.15 g, 96%). MS (ESI): 306.1 (M+H)⁺.

d) 2-[N-(N-benzyloxycarbonyl-L-leucinyl)]carbohydrazide

Following the procedure of Example 1(b), except substituting (1S)-1-benzyloxycarbonylamino-3-methyl- 1-(1,3,4oxadiazol-2-on-5-yl)butane for N-benzyloxycarbonyl-L-leucine methyl ester, the title compound was prepared as a white foam (0.097 g, 60%). MS (ESI): 338.2 (M+H)⁺.

e) 2-[N-(N-benzyloxycarbonylglycinyl)]-2′-[N′-(N-benzyloxycarbonyl-L-leucinyl)]carbohydrazide

To a solution of the compound of Example 1(d) (0.2 g, 0.593 mmol), N-benzyloxycarbonylglycine (0.137 g, 0.653 mmol), and 1-hydroxybenzotriazole (0.016 g, 0.119 mmol) in DMF (6mL) was added 1-(3-dimethylaminopropyl)-3-ethylcarbodiimide hydrochloride (0.125g, 0.653 mmol). After stirring at room temperature for 16 hours the solution was diluted with ethyl acetate and washed successively with saturated aqueous sodium bicarbonate, water and brine. The organic layer was dried (MgSO₄), filtered and concentrated. The residue was purified by column chromatography (silica gel; dichloromethane/methanol) to yield the title compound as a white solid (0.204 g, 65%). MS (ESI): 529.2 (M+H⁺).

### EXAMPLE 2

Preparation of (3RS)-I-(N-benzyloxycarbonyl-L-leucinyl)-3-[N-(4-phenoxybenzoyl)aminolpyrrolidin-4-one

a) 1-tert-butoxycarbonyl-3-pyrroline

To a solution of 3-pyrroline (5.0 g, 72.35 mmol) in CH₂Cl₂ (25 mL) at room was added di-tert-butyl dicarbonate (16.58 g, 75.97 mmol) in CH₂Cl₂ (50 mL). The reaction was stirred for 1 hour whereupon it was concentrated in vacuo to give the title compound which was used directly in the following step without further purification. ¹H NMR (200 MHz, CD₃OD) d 5.12 (m, 2H), 3.92 (m, 4H), 1.38 (s, 9H).

b) 1-tert -butoxycarbonyl-3,4-epoxypyrrolidine

To a solution of compound of Example 2(a) (5.0 g, 29.5 mmol) in CH₂Cl₂ (200 mL) was added NaHCO₃ (9.03 g, 118.2 mmol) and m-chloroperbenzoic acid (15.29 g, 88.6 mmol). The reaction was allowed to stir at room temperature overnight whereupon it was concentrated and filtered with petroleum ether. The petroleum ether layer was washed with saturated K₂CO₃ (2x), water, saturated brine, dried (MgSO₄), filtered and concentrated to give a clear colorless oil. Column chromatography of the oil (4:1 hexanes:ethyl acetate) gave the title compound which was used directly in the following step. ¹H NMR (200 MHz, CDCl₃) 3.85-3.20 (m, 6H), 1.43 (s,9H).

c.) trans-3-azido-1-tert-butoxycarbonyl-4-hydroxypyrrolidine

To a stirring solution of the compound of Example 2(b) (2.03 g, 10.96 mmol) in methanol:water (18 mL of an 8:1 solution) was added ammonium chloride (2.5 g, 10.96 mmol) and sodium azide (3.56 g, 54.8 mmol). The reaction was heated at 60° C. overnight whereupon it was diluted with petroleum ether, washed with pH 4 buffer, saturated sodium bicarbonate, saturated brine, dried (MgSO₄), filtered and concentrated to give 2.12 grams of the title compound which was carried onto the next step without further purification. ¹H NMR (400 MHz, CDCl₃) 4.21 (br s, 1H), 3.92 (br s, IH), 3.71-3.30 (m, 4H), 1.43 (s, 9H).

d) trans-3-amino-1-tert-butoxycarbonyl-4-hydroxypyrrolidine

To a solution of the compound of Example 2(c) (210 mg, 0.92 mmol) in CH₃OH (10 mL) was added 10% Pd on carbon. This mixture was stirred under an atmosphere of hydrogen until TLC analysis indicated the complete disappearence of the starting material. The reaction was filtered through a pad of celite with CH₂Cl₂ and concentrated to give 202 mg of the title compound which was used directly in the next step.

e) trans-(3RS,4RS)-l-tert-butoxycarbonyl-4-hydroxy-3-[N-(4-phenoxybenzoyl)amino]pyrrolidine

Following the procedure of Example 1(e), except substituting trans-3-amino-1-tert-butoxycarbonyl-4-hydroxypyrrolidine for 2-[N-(N-benzyloxycarbonyl-L-leucinyl)]carbohydrazide and 4-phenoxybenzoic acid for N-benzyloxycarbonylglycine, the title compound was prepared and was carried onto the next step.

f.) trans-(3RS,4RS)-4-hydroxy-3-[N-(4-phenoxybenzoyl)amino]pyrrolidine hydrochloride

To a solution of the compound of Example 2(e) (228 mg, 0.57 mmol) in dry EtOAc (5.0 mL) was bubbled HCl gas for approximately 5 minutes. The reaction was stirred until TLC analysis indicated the complete consumption of the starting material. The reaction was then concentrated in vacuo to give 168 mg (88%) of the title compound which was carried on to the next step.

g) trans-(3RS,4RS)-1-(N-benzyloxycarbonyl-L-leucinyl)-4-hydroxy-3-[N-(4-phenoxybenzoyl)amino]pyrrolidine

Following the procedure of Example 1(e), except substituting trans-(3RS,4RS)-4-hydroxy-3-[N-(4-phenoxybenzoyl)amino]pyrrolidine hydrochloride for 2-[N-(N-benzyloxycarbonyl-L-leucinyl) ]carbohydrazide and N-benzyloxycarbonyl-L-leucine for N-benzyloxycarbonylglycine, the title compound was prepared. MS (ESI): 546.3 (M+H)⁺, 568.2 (M+Na)⁺.

h) (3RS)-1-(N-benzyloxycarbonyl-L-leucinyl)-3-[N-(4-phenoxybenzoyl)amino]pyrrolidin-4-one

To a 0° C. solution of the compound of Example 2(g) (150 mg) in acetone (5 mL) was added Jones reagent dropwise until the brown color persisted. The reaction was allowed to warm to room temperature and stirred approximately 18 hours whereupon it was quenched with iso-propanol, diluted with EtOAc and washed sequentially with saturated K₂CO₃, water and saturated brine. The organic layer was dried (MgSO₄), filtered and concentrated. Column chromatography of the residue (2:1 EtOAc:hexanes) gave 49 mg of the title compound. MS (ESI): 544.2 (M+H)⁺.

### EXAMPLE 3

Preparation of (1S)-N-[2-[1-(N-benzyloxycarbonylamino)-3-methylbutyl]thiazol-4-ylcarbonyl]- N′-[N-(2-pyridinvlmethoxyearbonyl)-L-leucinyl]hydrazide

a) N-benzyloxycarbonyl-L-leucinamide

To a stirring solution of N-benzyloxycarbonyl-L-leucine (4.6 g, 17.3 mmol) in THF, cooled to −40° C., was added N-methylmorpholine (3.68 g, 36.4 mmol; 4.0 mL) and isobutyl chloroformate (2.37 g, 17.3 mmol; 2.25 mL). After stirring for 15 min, ammonia was bubbled through the solution for 5 min. The solution was warmed to room temperature, evaporated, and the residue was dissolved in ethyl acetate, washed with 0.1 N HCl, and saturated brine, then dried (MgSO₄), filtered and evaporated to dryness to give the title compound as a white solid (4.58 g, 100%).

b) N-benzyloxycarbonyl-L-leucinethioamide

A solution of the compound of Example 3(a) (4.58 g, 17.3 mmol) and Lawesson's reagent (4.21 g, 10.4 mmol) in THF was allowed to stir at room temperature for 16 hours. The solution was concentrated and the residue was purified by flash chromatography on 230-400 mesh silica gel, eluting with 1:3 EtOAc/hexanes, to provide the title compound as a pale yellow solid (3.74 g, 77%).

c) (1S)-1-benzyloxycarbonylamino-1-(4-carboethoxythiazol-2-yl)-3-methylbutane

The compound of Example 3(b) (2.20 g, 7.83 mmol) was dissolved in acetone (35 mL), cooled to −10° C., and ethyl bromopyruvate (1.68 g, 8.62 mmol, 1.08 mL) was added. After stirring for 1 hour, the solution was poured into methylene chloride/water, then into saturated aqueous NaHCO₃. The aqueous layer was extracted with methylene chloride and the combined organic layers were washed with saturated brine, dried (MgSO₄), filtered and concentrated. The residue was dissolved in methylene chloride, cooled to −20° C., pyridine (1.36 g, 17.2 mmol, 1.39 mL) and trifluororacetic anhydride (1.81 g, 8.62 mmol, 1.22 ML) were added. After stirring for 1 hour, the solution was washed with saturated squeous NaHCO₃ and saturated brine, then dried (MgSO₄), filtered, and concentrated. The residue was purified by flash chromatography on 90 grams of 230-400 mesh silica gel, eluting with 1:3 ethyl acetate/hexanes, to provide the title compound as a pale yellow oil (2.36 g, 80%). ¹H NMR (400 MHz, CDCl₃) d 8.08 (s, 1H), 7.38 (m, 5H), 5.42 (s, 3H), 5.23-5.07 (m, 3H), 4.42 (q, 2H), 2.01-1.62 (m, 3H), 1.41 (t, 3H), 0.99 (d, 6H).

d) (1S)-1-benzyloxycarbonylamino-1-(4-hydrazinocarbonylthiazol-2-yl)-3-methylbutane

The compound of Example 3(c) (2.16 g, 5.73 mmol) was dissolved in ethanol (60 mL) and hydrazine hydrate (2.87 g, 57.3 mmol, 2.8 mL) was added and the solution was heated at 75° C. for 1 hour. The solution was cooled and evaporated to dryness to provide the title compound as a pale yellow foam (2.01 g, 97%). 1H NMR (400 MHz, CDCl₃) d 8.35 (bs, IH), 8.03 (s, 1H), 7.37 (m, SH), 5.29 (d, IH), 5.14-5.09 (m, 3H), 4.07 (bs, 2H), 1.92-1.82 (m, 1H), 1.79-1.66 (m, 2H), 1.00 (d, 6H).

e) a-isocyanato-L-leucine methyl ester

L-leucine methyl ester hydrochloride (25 g, 0.14 mol) was dissolved in methylene chloride (450 mL), cooled to 0° C., and pyridine (43.5 g, 0.55 mol, 44.5 mL) was added, then a 1.93 M solution of phosgene in toluene (0.18 mol, 92.7 mL) was added slowly. After stirring at 0° C. for 2 hours, the mixture was poured into 0.5 N HCl (1400 mL) and ice (900 mL). The organic layer was washed with 0.5 N HCl (1400 mL) and ice (900 mL). The aqueous layers were extracted with methylene chloride (450 mL) and the combined organic layers were washed with saturated brine (1400 mL) and ice (900 mL), then dried (MgSO₄), filtered and concentrated. The residue was distilled (56-58° C.; 0.78 mmHg) to provide the title compound as a colorless liquid (20.4 g, 86%). ¹H NMR (250 MHz, CDCl₃) d 4.04 (dd, 1H), 3.82 (s, 3H), 1.92-1.72 (m, 1H), 1.69-1.62 (m, 2H), 0.96 (d, 3H), 0.94 (d, 3H).

f) N-(2-pyridinylmethoxycarbonyl)-L-leucine methyl ester

A solution of the compound of Example 3(e) (5.5 g, 32.3 mmol) and 2-pyridylcarbinol (3.5 g, 32.3 mmol) in toluene (35 mL) was heated at reflux for 24 hours. The solution was concentrated and the residue was purified by flash chromatography on 60 grams of 230-400 mesh silica gel, eluting with 30% ethyl acetate in hexanes, to provide the title compound as a pale yellow oil (8.06 g, 89%). MS (ESI): 281.2 (M+H)⁺.

g) N-(2-pyridinylmethoxycarbonyl)-L-leucine

To a stirring solution the compound of Example 3(f) (745 mg, 2.6 mmol) in THF (3 mL) was added 3 mL of water followed by LiOH.H₂O (120 mg, 2.86 mmol). The mixture was stirred for 30 minutes and then concentrated. The residue was redissolved in water (4 mL) and 3 N HCl was added (0.95 niL. The solution was lyophilized to yield a white solid (680 mg, 94%). MS (ESI): 267.2 (M+H)⁺.

h) (IS)-N-[2-[1 -(N-benzyloxycarbonylamino)-3-methylbutyl]thiazol4-ylcarbonyl]-N′-[N-(2-pyridinylmethoxycarbonyl)-L-leucinyl]hydrazide

Following the procedure of Example 1(e), except substituting (1S)-1-benzyloxycarbonylamino-1-(4-hydrazinocarbonylthiazol-2-yl)-3-methylbutane for 2-[N-(N-benzyloxycarbonyl-L-leucinyl)]carbohydrazide and N-(2-pyridinylmethoxycarbonyl)-L-leucine for N-benzyloxycarbonylglycine, the title compound was prepared as a white solid (125 mg, 65%). MS (ESI): 611.2 (M+H)⁺.

### EXAMPLE 4

Preparation of l-(N-benzyloxycarbonyl-L-leucinylamino)-3-(2-benzyloxvphenylsulfonyl)aminopropan-2-one

a) 1-amino-3-(N-benzyloxycarbonyl-L-leucinylamino)propan-2-ol 1,3-Diamino-propan-2-ol (6.75 g, 75 mmol) was dissolved in DMF (100 mL). Then 1-hydroxybenzotriazole hydrate (11.0 g, 81.5 mmol), N-benzyloxycarbonyl-L-leucine (20 g, 75.5 mmol), 1-(3-dimethylaminopropyl)-3-ethylcarbodiimide methiodide (15.5 g, 81.2 mmol), were added, and the reaction mixture was allowed to stir overnight. The DMF was then removed in vacuo and the reaction mixture was diluted with diethyl ether (150 mL) and MeOH (90 mL). Then 1M HCl in diethyl ether was added (1M, 100 mL) forming a gum, which was further extracted with diethyl ether (200 ml). The combined organics were concentrated in vacuo, then chromatographed (silica gel, 1: 10:89 trifluoroacetic acid; MeOH; dichloromethane) to yield the title compound. MS (ESI): 338.3 (M+H)⁺.

b) 2-benzyloxyphenylsulfonyl chloride

A small crystal of iodine was added to a slurry of magnesium powder (0.63 g, 26.25 mmol) and 2-benzyloxybromobenzene (Friesen, Richard W.; Sturino, Claudio F.; J. Org. Chem. 55; 9; 1990; 2572-2574, 6.0 g, 22.8 mmol) in THF (20 mL) and was heated to reflux for 1 hour. Then the reaction was cooled to 0° C. and SO₂Cl₂ (3.5 ml, 43.6 mmol) was added and the reaction was stirred for 2 hours at room temperature. The reaction mixture was then quenched with ice water and extracted with diethyl ether. The combined organics were then washed with saturated brine, dried (MgSO₄), filtered and concentrated to give a solid which was used in the next reaction without further purification. ¹H NMR (CDCl₃) d 8.0-6.8 (9H, m), 5.35 (3H, s).

c) 1-(N-benzyloxycarbonyl-L-leucinylamino)-3-(2-benzyloxyphenylsulfonyl)amino-propan-2-ol

The compound of Example 4(a) (0.4 g, 1 mmol) was dissolved in DMF (4 mL) and N-methylmorpholine (0.3 g, 0.35 mL, 3 mmol) was added. The compound of Example 4(b) (0.28 g, 1 mmol) was then added and the reaction was allowed to stir for 4 hours. The reaction mixture was concentrated in vacuo, then chromatographed on silica gel to yield the title compound. MS (ESI): 584.2 (M+H)⁺.

d) 1-(N-benzyloxycarbonyt-L-leucinylamino)-3-(2-benzyloxyphenyisulfonyI)amino-propan-2-one

Following the procedure of Example 2(h), except substituting 1-(N-benzyloxycarbonyl-L-leucinylamino]-3-(2-benzyloxyphenylsulfonyl)amino-propan-2-ol for trans-(3RS,4RS)-1-(N-benzyloxycarbonyl-L-leucinyl)-4hydroxy-3-[N-(4-phenoxybenzoyl)amino]pyrrolidine, the title compound was prepared as a white solid (35 mg, 70%). MS (ESI): 582.5 (M+H)⁺.

### EXAMPLE 5

Preparation of N-f2-(2-benzyloxvphenyl)thiazol-4-ylcarbonyll-N-[N-(3-pyridinylmethoxycarbonyl)-L-leucinyl]hydrazide

a) ethyl 2-aminothiazole-4-carboxylate hydrobromide

To a stirring suspension of thiourea (6.0 g, 78.8 mmol) in ethanol (80 mL) was added ethyl bromopyruvate (15.4 g, 78.8 mmol). The resulting solution was heated at 45° C. for 23 hours. The solution was cooled at 0° C. for 24 hours, and the crystals were collected by filtration and washed with cold ethanol to provide the title compound (15.8 g, 79%). ¹H NMR (400 MHz, CD₃OD) d 7.70 (s, 1H), 4.41 (q, 2H), 1.38 (t, 3H).

b) ethyl 2-bromothiazole-4-carboxylate

To a stirring suspension of the compound of Example 5(a) (I 2.15 g, 48 mmol) in 16% aqueous HBr (150 mL), cooled to 0° C., was added dropwise a solution of sodium nitrite (3.44 g, 49.8 mmol) in water (6 mL). After stirring for 35 min, copper (I) bromide (7.83 g, 54.6 mmol) and 16% aqueous HBr (60 mL) were added and the mixture was heated at 70° C. for 1 hour. The mixture was filtered and the filtrate was saturated with NaCl then extracted with ethyl acetate (2×170 mL). The combined extracts were dried (MgSO₄), filtered and evaporated to dryness. The residue was combined with the solid collected in the first filtration, heated at reflux in ethanol (500 mL) for 5 minutes, then filtered. To the filtrate was added 1.5 mL of 48% aqueous HBr and the solution was heated at reflux for 16 hours, then concentrated. The residue was partitioned between saturated aqueous NaHCO₃ and ethyl acetate. The organic layer was washed with saturated brine, dried (MgSO₄), decolorized with charcoal, filtered and concentrated to provide the title compound as a pale yellow solid (7.46 g, 75%). MS (ESI): 236.0 (M+H)⁺.

c) 2-benzyloxyphenylboronic acid

To a stirring solution of the compound of 2-benzyloxybromobenzene (15.2 g, 57.8 mmol) in THF (100 mL) at -78° C was added dropwise n-BuLi (23.1 mL, 2.5M in hexane, 57.8 mmol). The mixture stirred at −78° C. for 25 min when added via cannulation to a stirring solution of triisopropylborate (54.4 g, 289 mmol) in THF (100 mL) at −78° C. After warming to room temperature and stirring for 3 hours, the mixture was poured into 3N HCl (100 mL) and extracted with ethyl acetate (3×200 mL). The organic layers were combined, washed successively with water and brine, dried (MgSO₄), filtered and concentrated. The residue was purified by column chromatography (silica gel, ethyl acetate/hexane) to yield the title compound as a pale yellow solid (6.9 g, 30.3 mmol). ¹NMR (400 MHz, CDCl₃) d 7.90 (d, 1H), 742 (m, 6H), 7.07 (t, IH), 7.02 (d, 1H), 6.05 (s, 2H), 5.16 (s, 2H).

d) ethyl 2-(2-benzyloxyphenyl)thiazole-4-carboxylate

To a stirring solution of the compound of Example 5(b) (4.0 g, 16.9 mmol), the compound of Example 5(d) (4.29 g, 18.8 mmol), tetrakis(triphenylphosphine)palladium(0) (0.65 g, 0.57 mmol) in dimethoxyethane (60 mL) was added cesium fluoride (8.58 g, 56.5 mmol) and the mixture was heated at 85 ° C for 16 hours. Tetrakis(triphenylphosphine)palladium(0) (0.65 g, 057 mmol) was added and heating at 85° C. was continued for 5 hours. The mixture was diluted with water (60 mL) and extracted with ethyl acetate (2×120 mL). The combined extracts were washed with saturated aqueous NaHCO₃ and saturated brine, dried (MgSO₄), filtered and concentrated. The residue was purified by flash chromatography on 180 grams of 230-400 mesh silica gel, eluting with 15% ethyl acetate in hexanes, to provide the title compound as a white solid (3.22 g, 56%). MS (ESI): 340.3 (M+H)⁺.

e) 2-(2-benzyloxyphenyl)thiazol-4-ylcarbonylhydrazide

Following the procedure of Example 3(d), except substituting ethyl 2-(2-benzyloxyphenyl)thiazole-4-carboxylate for (1S)-1-benzyloxycarbonylamino-1-(4-carboethoxythiazol-2-yl)-3-methylbutane, the title compound was prepared as a white solid. MS (ESI): 326.2 (M+H)⁺.

f) N-[2-(2-benzyloxyphenyl)thiazol-4-ylcarbonyl]-N′[N-(3-pyridinylmethoxycarbonyl)-L-leucinyl]hydrazide

Following the procedure of Example 3(e)-3(h), except substituting 3-pyridylcarbinol for 2-pyridylcarbinol in step (f), N-(3-pyridinylmethoxycarbonyl)-L-leucine for N-(2-pyridinylmethoxycarbonyl)-L-leucine and 2-(2-benzyloxyphenyl)thiazol-4-ylcarbonylhydrazide for (1S)-1-benzyloxycarbonylamino-1-(4-hydrazinocarbonylthiazol-2-yl)-3-methylbutane in step (h), the title compound was prepared as a white solid (93.8 mg, 53%). MS (ESI): 574.3 (M+H)⁺.

### EXAMPLE 6

Preparation of (1S)-N-[2-[1-(N-benzyloxycarbonylamino)-3-methylbutyl]thiazol-4-ylcarbonyl]- N′-[N-(3-pyridinylmethoxycarbonyl)-L-leucinyl]hydrazide

Following the procedure of Example 3(a)-3(h), except substituting 3-pyridylcarbinol for 2-pyridylcarbinol (f), the title compound was prepared as a white solid (63 mg, 42%). MS (ESI): 611.5 (M+H)⁺.

### EXAMPLE 7

Preparation of 1-[N-(4-monpholinocarbamoyl)-L-leucinylamino]-3-(4-phenoxyphenylsulfonyl)amino-propan-2-one

a) ]-(N-benzyloxycarbonyl-L-leucinylamino)-3-(4-phenoxyoxyphenylsulfonyl)amino-propan-2-ol

Following the procedure of Example 4(a)-4(c), except substituting 4-phenoxybromobenzene for 2-benzyloxybromobenzene in step (a), the title compound was prepared. MS (ESI): 570.1 (M+H⁺).

b) 1-(L-leucinylamino)-3-(4-phenoxyphenylsulfonyl)aminopropan-2-ol

The compound of Example 7(a) (5.0 g, 8.79 mmol) and 10% Pd/C (1.03 g) in EtOH (140 ml) and was allowed to stir under a baloon of hydrogen gas for 4 hours. The reaction mixture was filtered through Celite, concentrated and was used in the next reaction without further purification. MS (ESI): 436 (M+H)⁺.

c) 1-[N-(4-morpholinocarbamoyl)-L-leucinylamino]-3-(4-phenoxyphenylsulfonyl)amino-propan-2-ol

The compound of Example 7(b) (0.24 g, 0.5 mmol), N-methylmorpholine (0.15 g, 0.16 mL, 1.5 mmol), and morpholine-4-carbonyl chloride (J. Chem. Soc. 1947; 307, 313; 0.076 g, 0.5 mmol) in DMF (3 mL). The reaction mixture was allowed to stir overnight, then concentrated and chromatographed (silica gel, 4:1 EtOAc: hexanes) to yield the title compound. MS (ESI): 549.4 (M+H)⁺.

d) 1-[N-(4-morpholinocarbamoyl)-L-leucinylamino]-3-(4-phenoxyphenylsulfonyl)amino-propan-2-one

Following the procedure of Example 2(h), except substituting 1-[N-(4-morpholinocarbamoyl)-L-leucinylamino]-3-(4-phenoxyphenylsulfonyl)amino-propan-2-ol for trans-(3RS,4RS)-1 -(N-benzyloxycarbonyl-L-leucinyl)-4-hydroxy-3-[N-(4-phenoxybenzoyl)amino ]pyrrolidine, the title compound was prepared. MS (ESI): 547.3 (M+H)⁺.

### EXAMPLE 8

Preparation of N-[2-(1-naphthyl)thiazol-4-ylcarbonyl]-N′-[N-(4-pyridinylmethoxycarbonyl)-L-leucinyl]hydrazide

Following the procedure of Example 5(a)-5(b) and 5(d)-5(f), except substituting 1-naphthyl boronic acid for 2-benzyloxyphenyl boronic acid in step (d) and 4-pyridylcarbinol for 3-pyridylcarbinol in step (f), the title compound was prepared as a white solid (0.094 g, 58%). MS (ESI): 518.4 (M+H)⁺.

### EXAMPLE 9

Preparation of N-r2-(2-benzyloxyphenyl)thiazol-4-ylcarbonyll-N!-(N-pyrazinecarbonyl-L-leucinyl)hydrazide

a) N-[2-(2-benzyloxyphenyl)thiazol-4-ylcarbonyl]-N′(N-tert-butoxycarbonyl-L-leucinyl)hydrazide

Following the procedure of Example 5(a)-5(f), except substituting N-tert-butoxycarbonyl-L-leucine for N-(3-pyridinylmethoxycarbonyl)-L-leucine in step (f), the title compound was prepared as a white solid (1.015 g, 94%). MS (ESI): 539.1 (M+H)⁺.

b) N-[2-(2-benzyloxyphenyl)thiazol-4-ylcarbonyl]-N′-(L-leucinyl)hydrazide

To a stirring solution of the compound of Example 9(a) (1.012 g, 1.88 mmol) in dichloromethane (10 mL) was added trifluoroacetic acid (2 mL). After stirring at room temperature for 2 hours, the solution was concentrated and the residue dissolved in ethyl acetate. The solution was washed successively with saturated aqueous sodium bicarbonate and saturated brine. The organic layer was dried (MgSO₄), filtered and concentrated to yield the title compound as a white foam (0.766 g, 93%). MS (ESI): 439.3 (M+H)⁺.

c) N-[2-(2-benzyloxyphenyl)thiazol-4-ylcarbonyll-N′-(N-pyrazinecarbonyl-L-leucinyl)hydrazide

Following the procedure of Example 1(e), except substituting N-[2-(2-benzyloxyphenyl)thiazol-4-ylcarbonyl]-N′-(L-leucinyl)hydrazide for 2-[N-(N-benzyloxycarbonyl-L-leucinyl)carbohydrazide and pyrazinecarboxylic acid for N-benzyloxycarbonylglycine, the title compound was prepared as a white solid (0.146 g, 94%). MS(ESI): 545.4 (M+H)⁺.

### EXAMPLE 10

Preparation of N-FN-(1-benzyl-5-methylimidazol-4-ylcarbonyl)-L-leucinyl]-N′-[2-(1-naphthyl)thiazol-4-ylcarbonyl]hydrazide

a) N-(N-tert-butoxycarbonyl-L-leucinyl)-N′-[2-(1-naphthyl)thiazol-4-ylcarbonyl]hydrazide

Following the procedure of Example 5(a)-5(b) and 5(d)-5(f), except substituting 1-naphthyl boronic acid for 2-benzyloxyphenyl boronic acid in step (d) and N-tert-butoxycarbonyl-L-leucine for N-(3-pyridinylmethoxycarbonyl)-L-leucine in step (f), the title compound was prepared as a white solid (2.2 g, 96%). MS (ESI): 483.2 (M+H)⁺.

b) N-[N-(I-benzyl-5-methylimidazol-4-ylcarbonyl)-L-leucinyl]-N′-[2-(1-naphthyl)thiazol-4-ylcarbonyl]hydrazide

Following the procedure of Example 9(b)-9(c), except substituting N-(N-tert-butoxycarbonyl-L-leucinyl)-N′-[2-(1-naphthyl)thiazol-4-ylcarbonyl]hydrazide for N-[2-(2-benzyloxyphenyl)thiazol-4-ylcarbonyl]-N′-(N-tert-butoxycarbonyl-L-leucinylthydrazide in step (b) and 1-benzyl-5-methylimidazole-4-carboxylic acid for pyrazinecarboxylic acid in step (c), the title compound was prepared as a white solid (0.115 g, 75%). MS (ESI): 581.1 (M+H)⁺.

### EXAMPLE 11

Preparation of (3RS)-3-rN-(3-benzyloxybenzoyl)-L-leucinylamino]tetrahydrofuran-4-one

a) trans-3-azido-4-hydroxytetrahydrofuran

3,4-epoxytetrahydrofuran (9 g, 105 mmol) was added to a stirred solution of sodium azide (27 g, 415 mmol) and ammonium chloride (9 g, 159 mmol) in aqueous methanol (95%, 200 mL). The reaction was heated to 75° C. and stirred for 20 hours. The reaction was cooled, filtered and evaporated under reduced pressure. The residue was diluted with water and extracted with ethyl acetate, dried (MgSO₄), filtered and evaporated under reduced pressure to afford the title compound as a colorless oil (10 g, 74%). ¹H NMR d (CDCl₃) 4.32 (m, 1H), 4.09 (dd, 1H, J=4.8, 9.9 Hz), 3.99 (dd, 1H, J=4.3, 10.1 Hz), 3.94 (m, 1H), 3.81 (dd, IH, J =2.1, 9.9 Hz), 3.73 (dd, IH, J =1.8, 10.1 Hz), 2.72 (d, IH, J =4.6 Hz).

b) trans-3-amino-4-hydroxytetrahydrofuran hydrochloride

A mixture of the compound of Example 1 1(a) (10 g, 77 mmol) and 10% palladium on carbon (1 g) in ethanol (150 mL) was stirred under an atmosphere of hydrogen (35 psi) for 12 hours. The mixture was filtered and treated with 100 ml of ethanolic HCl to afford, after evaporation under reduced pressure, the title compound as a brown solid (10.5 g, 97% yield). m.p. 132° C. ¹H NMR d (d₆ DMSO) 8.37 (s, 3H), 4.13 (m, 1H), 3.84 (dd, 1H, J=4.9 and 14.3), 3.76 (dd, IH, J =5.5, 10.0 Hz), 3.58 (dd, 1H, J=2.7, 10.0 Hz), 3.34 (m, 3 H).

c) trans-(3RS, 4RS)-3-[N-(tert-butoxycarbonyl)-L-leucinylamino]-4-hydroxytetrahydrofuran

Trimethylacetyl chloride (3.5 ml, 29 mmol) was added to a stirred solution of N-tert-butoxycarbonyl-L-leucine (7.3 g, 31 mmol) and N,N-diisopropylethylamine (9 ml, 52 mmol) in dichloromethane (200 mL). After 1 hour, the compound of Example I l(b) (4 g, 28 mmol) was added and the mixture was allowed to stir overnight. The reaction mixture was poured into water and extracted with dichloromethane. The combined organic layers were washed with 0.5N HCl, saturated sodium bicarbonate and saturated brine, then dried (MgSO₄) and filtered. Evaporation under reduced pressure afforded the title compound as a yellow foam (5 g, 44%). ¹H NMR d (CDCl₃) 8.08 (d, 0.5H, J=4.8 Hz), 7.89 (d, 0.5H, J=7.4 Hz), 6.20 (d, 0.5H, J=8.3 Hz), 6.09 (d, 0.5H, J=8.7 Hz), 4.81 (d, 1H, J =16.0Hz), 4.40 (m, 2H), 4.20 (m, 2H), 3.77 (m, 2H), 1.60 (m, 3H), 1.50 (s, 9H), 0.92 (m, 6H).

d) trans-(3RS, 4RS)-3-(L-leucinylamino)-4-hydroxytetrahydrofuran trifluoroacetate salt

Following the procedure of Example 9(b), except substituting ) trans-(3RS)-3-[N-(tert-butoxycarbonyl)-L-leucinylamino]-4-hydroxytetrahydrofuran for N-[2-(2-benzyloxyphenyl)thiazol-4-ylcarbonyl]-N′-(N-tert-butoxycarbonyl-L-leucinyl)hydrazide, the title compound was prepared as a white gum (2.6 g, 100%). ¹H NMR d (MeOD) 4.18 (m, 2H), 4.08 (m, 2H), 3.97 (m, 2H), 3.86 (apparent t, 2H, J =7.1 Hz), 3.69 (dd, 2H, J =1.6, 7.4 Hz), 1.68 (m, 3H), 0.99 (d, 6H, J=2.1 Hz).

e) methyl 3-benzyloxybenzoate

To a suspension of NaH (0.395 g, 9.87 mmol, 60% in mineral oil) in DMF (20 mL) was added methyl 3-hydroxybenzoate (1.0 g, 6.58 mmol). After stirring for 15 minutes at room temperature, benzyl bromide (1.1 g, 6.58 mmol) was added. After stirring at room temperature for 3 hours, the solution was partitioned between ethyl acetate and water. The organic layer was washed with water (2×75 mL), saturated aqueous sodium bicarbonate, and brine, then dried (MgSO₄), filtered and concentrated to yield the title compound as an off-white solid (1.013 g, 4.2 mmol). ¹H NMR (400 MHz, CDCl₃) d 7.67 (m, 2H), 7.48-7.34 (m. 6H), 7.19 (m, 1H), 5.12 (s, 2H), 3.95 (s, 3H).

f) 3-benzyloxybenzoic acid

To a solution of the compound of Example I I(e) (0.400 g, 1.65 mmol) in THF (2 mL) and water (2 mL) was added lithium hydroxide monohydrate (0.076 g, 1.82 mmol). After stirring at reflux for 5 hours, the solution was partitioned between ethyl acetate and 3N HCl. The organic layer was washed with brine, dried (MgSO₄), filtered and concentrated to yield a white solid (0.355 g, 1.56 mmol). ¹H NMR (400 MHz, CD₃OD) d 7.58 (m, 2H), 7.36-7.24 (m. 6H), 7.10 (m, 1H), 5.04 (s, 2H).

g) trans-(3RS, 4RS)-3-[N-(3-benzyloxybenzoyl)-L-leucinylamino]-4-hydroxytetrahydrofuran

The compound of Exmaple 1 1(f) (251 mg, 1.0 mmol) was added to a stirring solution of the compound of Example 11(d) (329 mg, 1.0 mmol), diethyl cyanophosphonate (0.16 ml, 1.0 mmol) and triethylamine (0.3 ml, 2.1 mmol) in dichloromethane (5 mL). The reaction was allowed to stir for I hour then diluted with ether. The organic layer was washed with IN hydrochloric acid, sodium bicarbonate and saturated brine, then dried (MgSO₄) and filtered. Evaporation of the solvent gave the title compound as a colorless oil (302 mg, 71%). ¹H NMR d (CDCl₃) 8.17 (d, 0.5H, J=5.1 Hz), 8.03 (d, 0.5H, J=7.5 Hz), 7.87 (d, 0.5H, J =7.8 Hz), 7.56 (d, 0.5H, J =7.5 Hz), 7.46-6.80 (m, 9H), 5.08 (appd, 2H, J =10.IHz), 5.07-4.70 (m, 1H), 4.45 (brs, 1H), 4.17 (brs, IH), 4.12-3.80 (m, 4H), 3.70-3.50 (m, 4H), 1.81-1.62 (m, 3H), 0.93-0.88 (m, 6H). MS (ESI): 425 (M+H)⁺.

h) (3RS)-3-[N-(3-benzyloxybenzoyl)-L-leucinylamino]tetrahydrofuran-4-one

Dess-Martin periodinane (500 mg, 1.2 mmol) was added to a stirring solution of the compound of Example 11(g) (280 mg, 0.7 mmol) in dichloromethane (10 mL). After 1 hour, ether was added followed by sodium thiosulfate (570 mg, 3.6 mmol). After an additional 15 minutes the reaction was washed with saturated sodium bicarbonate and saturated brine, then dried (MgSO₄) and filtered. Evaporation of the solvent gave the title compound as a white foam (270 mg, 93%). ¹H NMR d (CDCl₃) 7.92 (d, 0.5H, J=6.7 Hz), 7.83 (d, 0.5H, J 6.7 Hz), 7.48-7.04 (m, 10H), 5.04 (app d, 2H, J =4.2 Hz), 4.99-4.81 (m, 1H), 4.48-3.68 (m, 5H), 1.81-1.62 (m, 3H), 0.93-0.84 (m, 6H). MS (ESI): 423 (M+H)⁺.

### EXAMPLE 12

Preparation of N-[2-[N-cvclopropyl-N-(2-methylpropyl)amino]thiazol-4-ylcarbonyl]-N′-[N-(5-methyl-2-phenyloxazol-4ylcarbonyl)-L-leucinyl]hydrazide

a) N-cyclopropylisobutylamine

Cyclopropylamine (12.0 mL, 173 mmol) and isobutyraldehyde (15.8 mL, 173 mmol) were dissolved in methylene chloride (50 mL) and stirred at room temperature for one hour. The solution was then cooled to 0° C. and sodium triacetoxyborohydride (73 g, 346 mmol) was added with 400 mL methylene chloride. The solution mixture was stirred for 4 hours and then washed with sodium bicarbonate (5% aqueous solution). The organic phase was dried over MgSO₄, filtered and concentrated to afford the title compound as a colorless liquid (14.0 g, 71%). MS (ESI): 113.7 (M+H)⁺.

b) N-cyclopropyl-N-(2-methylpropyl)-N′-benzoylthiourea

The compound of Example 12(a) (14.0 g, 123 mmol) was dissolved in chloroform (100 mL) and benzoyl isothiocyanate (20 g, 123 mmol, 18 mL) was added. After stirring 45 minutes at room temperature, the solution was concentrated to provide the title compound as a yellow solid (29 g, 85%). MS (ESI): 257.1 (M+H)⁺.

c) N-cycldpropyl-N-(2-methylpropyl)thiourea

The compound of Example 12(b) (29 g, 105 mmol) was dissolved in methanol (100 mL) and water (100 mL), potassium carbonate (43 g, 315 mmol) was added and the solution was heated at reflux overnight. The reaction mixture was concentrated, redissolved in ethyl acetate, washed with sodium bicarbonate, water and dried over MgSO₄, filtered and concentrated to afford the title compound as a yellow solid (13.42 g, 75%). MS (ESI): 172.9 (M+H)⁺.

d) ethyl 2-[N-cyclopropyl-N-(2-methylpropyl)amino]thiazole-4-carboxylate

The compound of Example 12(c) (1 3.42 g, 77.7 mmol) was dissolved in ethanol (30 mL) upon heating. The solution was cooled to room temperature and ethylbromopyruvate (9.7 mL, 77.7 mmol) was added. The reaction mixture was heated at reflux for 30 minutes, then concentrated. The residue was partitioned between ethyl acetate and saturated aqueous sodium bicarbonate. The aqueous phase was extracted with ethyl acetate and the combined organic phases were washed with saturated brine, dried (MgSO₄), filtered and concentrated to give a yellow oil. The crude product was passed through silica gel eluting with ethyl acetate/hexane (1:3) to provide the title compound as a yellow oil (9.9 g, 48%). MS (ESI): 269.4 (M+H)⁺.

e) 2-[N-cyclopropyl-N-(2-methylpropyl)amino]thiazol-4-ylcarbonylhydrazide

Following the procedure of Example 3(d), except substituting ethyl 2-[N-cyclopropyl-N-(2-methylpropyl)aminolthiazole-4-carboxylate for (1S)-1-benzyloxycarbonylamino-1-(4-carboethoxythiazol-2-yl)-3-methylbutane, the title compound was prepared as a white solid (7.5 g, 80%). MS (ESI): 255.2 (M+H)⁺.

f) N′-(N-tert-butoxycarbonyl-L-leucinyl)-N′-[2-[N-cyclopropyl-N-(2-methylpropyl)amino]thiazol-4-ylcarbonyl]hydrazide

Following the procedure of Example I (e), except substituting 2-[N-cyclopropyl-N-(2-methylpropyl)amino]thiazol-4-ylcarbonylhydrazide for 2-[N-(N-benzyloxycarbonyl-L-leucinyl)]carbohydrazide and N-tert-butoxycarbonyl-L-leucine for N-benzyloxycarbonylglycine, the title compound was prepared as a white solid (7.5 g (100%). MS (ESI): 468.3 (M+H)⁺.

g) N-[2-[N-cyclopropyl-N-(2-methylpropyl)amino]thiazol-4-ylcarbonyl]-N′-[N-(5-methyl-2-phenyloxazol-4-ylcarbonyl)-L-leucinyl]hydrazide

Following the procedure of Example 9(b)-9(c), except substituting N′-(N-tert-butoxycarbonyl-L-leucinyl)-N′-[2-[N-cyclopropyl-N-(2-methylpropyl)aminouthiazo1-4-ylcarbonyl]hydrazide for N-[2-(2-benzyloxyphenyl)thiazol-4-ylcarbonyl]-N′-(N-tert-butoxycarbonyl-L-leucinyl)hydrazide in step (b) and 5-methyl-2-phenyloxazole-4-carboxylic acid for pyrazinecarboxylic acid in step (c), the title compound was prepared as a white solid (340 mg,). MS (ESI): 553.4 (M+H)⁺.

### EXAMPLE 13

Preparation of 1-[3-(2-pyridinvl)phenylacetylaminol-3-[N-(2-thiophenecarbonyl)-L-leucinylaminolpropan-2-one

a) methyl 3-(trifluoromethylsulfonyloxy)phenylacetate

To an oven-dried flask under Argon atmosphere containing sodium hydride (2.54 g, 60% dispersion in mineral oil, 63.5 mmol) was added anhydrous pentane (20 mL). The slurry was allowed to stir for 5 minutes, allowed to settle, most of the pentane was removed, and anhydrous THF (40 mL) was added. To this suspension was added a solution of methyl 3-hydroxyphenylacetate (9.99 g, 60.1 mmol) in anhydrous THF (20 mL) and the reaction was allowed to stir at room temperature for 20 minutes. To this mixture was then added a solution of N-phenyltrifluoromethanesulfonimide (22.53 g, 63.1 mmol)) in anhydrous THF (40 mL) and the reaction was allowed to stir at room temperature until TLC analysis indicated the complete consumption of starting material (1.5 h). The reaction was quenched by the addition of H₂O (10 mL), concentrated to one half original volume, then diluted with CHCl₃ (200 mL) and washed with H₂0. The aqueous layer was washed with fresh CHCl₃ (50 mL), the combined organic layers were washed with 10% Na₂CO₃, water, and saturated brine, then dried (MgSO4), filtered and concentrated. Column chromatography of the residue (silica gel, 5:95 EtOAc: hexanes, then 10:90 EtOAc: hexanes) gave 17.47 grams of the title compound. ¹H NMR (400 MHz, CDCl₃) 7.42 (m, 1H), 7.31-7.19 (m, 3H), 3.72 (s, 3H), 3.68 (s, 2H).

b) methyl 3-(2-pyridyl)phenylacetate

To a solution of the compound of Example 13(a) (6.86 g, 23.0 mmol) in anhydrous dioxane (100 mL) was added 2-pyridyltributylstannane (8.89 g, 24.1 mmol), LiCl (2.94 g, 69.3 mmol), 2,6-di-tert-butyl-4-methylphenol (a few crystals), and Pd(PPh₃)₄ (632.1 mg, 0.55 mmol). The reaction was protected from light with foil and heated at reflux overnight. The reaction was allowed to cool to room temperature and was concentrated. Column chromatography of the residue (silica gel, 1:3 EtOAc: hexanes, then 1:2 EtOAc: hexanes) gave 3.85 grams of the title compound. MS (ESI): 228.1 (M+H)⁺.

c) 3-(2-pyridyl)phenylacetic acid

To a solution of the compound of Example 13(b) (3.8 g, 16.7 mmol) in THF (50 mL) was added a solution of LiOH.H₂O (780.2 mg, 18.6 mmol) in water (10 mL). The reaction was allowed to at room temperature until TLC analysis indicated the complete consumption of starting material (2 hours). The reaction mixture was concentrated to remove THF, then neutralized to pH 7 by the addition of 1N HCl, diluted with brine (50 mL), and washed with CHCl₃ (100 mL) The aqueous layer was readjusted back to pH 7 by the addition on IN NaOH and washed with fresh CHCl₃ (100 mL). After repeating this procedure once more, the organic layers were combined, dried (MgSO₄), filtered and concentrated to give 3.79 grams of the title compound. MS (ESI): 214.3 (M+H)⁺.

d) 1-[N-(tert-butoxycarbonyl)-L-leucinylamino]-3-[3-(2-pyridinyl)phenylacety]amino]propan-2-ol 1,3-Diaminopropan-2-ol (5.61 g, 22.5 mmol) was dissolved in DMF (36 mL). Then, 1-hydroxybenzotriazole hydrate (3.34 g, 24.75 mmol), N-tert-butoxycarbonyl-L-leucine (5.61 g, 22.5 mmol), 1-(3-dimethylaminopropyl)-3-ethylcarbodiimide methiodide (4.73 g, 24.75 mmol) were added, and the reaction mixture was stirred for 4 hours. The compound of Example 13(c) (1.68 g, 7.875 mmol) was added, followed by 1-hydroxybenzotriazole hydrate (1.276 g, 9.45 mmol) and 1-(3-dimethylaminopropyl)-3-ethylcarbodiimide methiodide (1.81 g, 9.45 mmol), and the reaction was stirred an additional 12 hours. The reaction mixture was concentrated in vacuo, then chromatographed on silica gel to yield the title compound as a white solid (1.70 g, 43%). MS (ESI): 499.3 (M+H)⁺.

e) 1-L-leucinylamino-3-[3-(2-pyridinyl)phenylacetylamino]propan-2-ol trifluoroacetate salt

Following the procedure of Example 9(b), except substituting 1-[N-(tert-butoxyfarbonyl)-L-leucinylaminof-3-[3-(2-pyridinyl)phenylacetylamino]propan-2-ol for N-[2-(2-benzyloxyphenyl)thiazol-4-ylcarbonyll-N′(N-tert-butoxycarbonyl-L-leucinyl)hydrazide, the title compound was prepared and was used in the next step without further purification. MS (ESI): 399.2 (M+H)⁺.

f) 1-[3-(2-pyridinyl)phenylacetylamino]-3-[N-(2-thiophenecarbonyl)-L-leucinylamino]propan-2-ol

1-(3-dimethylaminopropyl)-3-ethylcarbodiimide methiodide (0.138 g, 0.722 mmol) was added to a solution of the compound of Example 13(e) (0.6 mmol), N,N-diisopropylethylamine (0.23 g, 0.315 mL, 1.81 mmol), 1-hydroxybenzotriazole hydrate (0.097 g, 0.722 mmol), and 2-thiophenecarboxylic acid (0.077 g, 0.6 mmol) in DMF (10 mL). The reaction mixture was allowed to stir overnight, then was washed with saturated brine/EtOAc. The combined organic layers were dried (MgSO₄), filtered, concentrated, and chromatographed on silica gel to yield the title compound as a white foam (0.15 g, 49%). MS (ESI): 509.3 (M+H)⁺.

g) 1-[3-(2-pyridinyl)phenylacetylaminol-3-[N-(2-thiophenecarbonyl)-L-leucinylamino]propan-2-one

Following the procedure of Example 1(h), except substituting 1-[3-(2-pyridinyl)phenylacetylamino]-3-[N-(2-thiophenecarbonyl)-L-leucinylamino]propan-2-ol for trans-(3RS, 4RS)-3-[N-(3-benzyloxybenzoyl)-L-leucinylamino}-4-hydroxytetrahydrofuran, the title compound was prepared as a white solid (70 mg, 64%). MS (ESI): 507.4 (M+H)⁺.

### EXAMPLE 14

Preparation of (3S)-3-[N-(benzothiazol-6-ylcarbonyl)-L-leucinylamino]-1-[3-(2-pyridinyl)phenylacetylamino]butan-2-one

a) N-benzyloxycarbonyl-L-alanyl bromomethyl ketone

Isobutyl chloroformate (2.90 g, 21.2 mmol, 2.74 mL) was added dropwise to a solution of N-benzyloxycarbonyl-L-alanine (4.7 g, 21.2 mmol) and N-methylmorpholine (2.14 g, 21.2 mmol, 2.32 mL) in THF (40 mL) at -40 degrees C. The reaction was allowed to stir for 15 minutes, then was filtered and washed with ether. Diazomethane prepared from 12 grams of 1-methyl-3-nitro-nitroso-guanidine and 36 ml of 40% KOH in ether (300 ml) was added and the reaction was placed in a refrigerator overnight (0° C.). 30% HBr/AcOH (14 ml) was added dropwise to the crude reaction mixture and was allowed to stir for 5 minutes. The solution was washed with aqueous citric acid (2×50 mL), saturated aqueous sodium bicarbonate (3×150 mL), then saturated brine (100 mL). The combined organics were dried (MgSO₄), filtered and concentrated in vacuo to give the title compound as a solid which was used in the next step without purification. MS (ESI): 360.3 (M+H)⁺.

b) N-benzyloxycarbonyl-L-alanyl azidomethyl ketone

The compound of Example 14(a) (1.5 g, 5 mmol) was dissolved in DMF (10 mL), then sodium azide (0.39 g, 6 mmol) and potassium fluoride (0.58 g, 7.5 mmol) was added and the reaction was allowed to stir overnight. The reaction was partitioned between EtOAc and water, then the combined organic extracts were dried (MgSO4), filtered, concentrated in vacuo, then chromatographed (2-5% MeOH, methylene chloride, silica gel) to provide the title compound as a white solid (0.5 g, 38%). 1R (thin film): 2106.4 cm⁻¹.

c) (3S)-1-azido-3-benzyloxycarbonylaminobutan-2-ol

The compouund of Example 14(b) (0.5, 1.9 mmol) was dissolved in MeOH (10 mL) and sodium borohydride (0.144 g, 3.8 mmol) was added at 10° C. and the reaction was allowed to stir for 15 minutes. The reaction was quenched with water (10 mL) and was extracted with EtOAc (25 mL). The combined organic extracts were dried (MgSO₄), filtered and concentrated to give the title compound which was used without further purification (0.5 g, 100%).

d) (3S)-1-amino-3-benzyloxycarbonylaminobutan-2-ol

The compound of Example 14(c) (0.5 g, 1.9 mmol) was dissolved in MeOH (7.5 mL) and triethylamine (0.72 g, 7.1 mmol, 1.0 mL), 1,3-propanedithiol (1.08 g, 10 mmol, 1.07 mL) was added and the reaction was allowed to stir overnight, concentrated in vacuo, then the white solid was washed with hexane providing the title compound which was used in the next reaction without further purification. MS (ESI): 239.3 (M+H)⁺.

e) (3S)-3-benzyloxycarbonylamino-]-[3-(2-pyridinyl)phenylacetylamino]butan-2-ol

The compound of Example 14(d) (0.452 g, 1.9 mmol) and the compound of Example 13(c) (0.4 g, 1.9 mmol) were dissolved in DMF (15 mL), 1-hydroxybenzotriazole hydrate (0.27 g, 2 mmol) and 1-(3-dimethylaminopropyl)-3-ethylcarbodiimide methiodide (0.38 g, 2 mmol) were added, and the reaction mixture was allowed to stir overnight. The reaction was partitioned between EtOAc and I N NaOH, the combined organic layers were dried (MgSO₄), filtered and concentrated to give the title compound (0.33g, 40%). MS (ESI): 434.2 (M+H)⁺.

f) (3S)-3-amino-I-[3-(2-pyridinyl)phenylacetylamino]butan-2-ol

Following the procedure of Example 7(b), except substituting (3S)-1 -[3-(2-pyridinyl)phenylacetylamino]-3-benzyloxycarbonylaminobutan-2-ol for 1-(N-benzyloxycarbonyl-L-leucinylamino)-3-(4-phenoxyoxypheny]sulfonyl)amino-propan-2-ol, the title compound was prepared and was used in the next reaction without further purification. MS (ESI): 300.3 (M+H)⁺.

g) (3S)-3-tert-butoxycarbonyl-L-leucinylamino-1-[3-(2-pyridinyl)phenylacetylaminojbutan-2-ol

The compound of Example 14(f) (0.28 g, 0.75 mmol) was dissolved in DMF (10 mL). HBTU (0.3 g, 0.8 mmol), N-tert-butoxycarbonyl-L-leucine (0.2 g, 0.8 mmol), N-methylmorpholine (0.34 g, 3.37 mmol, 0.37 mL) were added, and the reaction mixture was allowed to stir overnight. The reaction mixture was concentrated in vacuo, then chromatographed on silica gel to yield the title compound as a white solid. MS (ESI): 513.2 (M+H)⁺.

h) (3S)-3-(L-leucinylamino)-]-[3-(2-pyridinyl)phenylacetylamino]butan-2-ol

The compound of Example 14(g) (2.0 g, 3.9 mmol) was dissolved in methylene chloride (140 mL) and 4 M HCl in dioxane (85 mL) and allowed to stir at room temperature for 2 hours. Toluene (100 mL) was added and the reaction mixture was concentrated in vacuo to give the title compound which was used in the following step without further purification: MS (ESI): 413.2 (M+H)⁺.

i) (3S)-3-(benzothiazol-6-ylcarbonyl-L-leucinylamino)-1-[3-(2-pyridinyl)phenylacetylamino]butan-2-ol

Following the procedure of Example 14(g), except substituting (3S)-3-(L-leucinylamino)-1-[3-(2-pyridinyl)phenylacetylamino]butan-2-ol for (3S)-3-amino-1-[3-(2-pyridinyl)phenylacetylamino]butan-2-ol and benzothiazole-6-carboxylic acid for N-tert-butoxycarbonyl-L-leucine, the title compound was prepared and was used in the next reaction without further purification. MS (ESI): 574.3 (M+H)⁺.

j) (3S)-3-[N-(benzothiazol-6-ylcarbonyl)-L-leucinylamino]-l-[3-(2-pyridinyl)phenylacetylaminolbutan-2-one

Following the procedure of Example 11(h), except substituting (3S)-3-(benzothiazol-6-ylcarbonyl-L-leucinylamino)-1-[3-(2-pyridinyl)phenylacetylamino]butan-2-ol for trans-(3RS, 4RS)-3-[N-(3-benzyloxybenzoyl)-L-leucinylamino]-4-hydroxytetrahydrofuran, the title compound was prepared as a white solid (30.1 mg, 20%). MS (ESI): 572.3 (M+H)⁺.

### EXAMPLE 15

Preparation of N-[2-TN-cyclopropyl-N-(2-methylpropyl)amino]thiazol-4-ylcarbonyl]-N′-N-(7-methoxybenzofuran-2-ylcarbonyl)-L-b-cyclopropylalanyl]hydrazide

a) (S)-2-(N-tert-butoxycarbonylamino)-4-pentenoic acid

To a stirring solution of (S)-2-amino-4-pentenoic acid (6.0 g, 52.2 mmol) in 1,4-dioxane (105 mL), water (53 mL), and 1N NaOH (53 mL) at 0° C. was added di-tert-butyl-dicarbonate (12.5 g, 57.4 mmol). After stirring at 0° C. for 2 hours, the mixture was concentrated and the residue dissolved in water (75 mL). A layer of ethyl acetate was added and the aqueous layer was acidified to pH 3 with 0.3N KHSO4. The aqueous layer was extracted with ethyl acetate (2x) and the organic layers were combined, washed with water (2x), then dried (MgSO₄), filtered and concentrated to afford the title compound as a colorless oil (10.6 g, 95%). MS (ESI): 214.0 (M+H⁺).

b) methyl (S)-2-(N-tert-butoxycarbonylamino)-3-cyclopropylpropionate

A solution of the compound of Example 15(a) (10.6g, 49.5mmol) in ether (500 mL) was cooled to 0° C. Meanwhile, to a suspension of 1-methyl-3-nitro- I -nitrosoguanidine (36 g, 247 mmol) in ether (500 mL) was added 40% NaOH (700 mL) slowly with occasional swirling. After addition of the NaOH, the mixture was allowed to stand at 0° C. for 20 minutes. The aqueous layer was then removed and the organic layer was added dropwise, with swirling, to the acid solution. When the addition was complete the solution was allowed to stir for 20 minutes at 0° C. After 20 min, palladium acetate (1.0 g, 4.4 mmol) was added and the resulting , mixture was allowed to stir for an additional 15 minutes. The mixture was then concentrated and the procedure repeated on the residue to yield the title compound as a tan colored oil (9.8 g, 82%). ¹H NMR (400 MHz, CDCl₃) d 5.17 (d, 1H), 4.38 (m, 1H), 3.72 (s, 3H), 1.62 (t, 2H), 1.42 (s, 9H), 0.68 (m, 1H), 0.42 (m, 2H), 0.08 (m, 2H).

c) (S)-2-(N-tert-butoxycarbonylamino)-3-cyclopropylpropionic acid

To a stirring solution of the compound of Example 15(b) (7.5 g, 30.6 mmol) in THF (40 mL) and water (40 mL) was added lithium hydroxide monohydrate (1.4 g, 33.7 mmol). After heating at reflux for 16 hours, the solution was concentrated. The residue was dissolved in ethyl acetate and washed with 1N HCl. The organic layer was washed with brine, dried (MgSO₄), filtered and concentrated to yield the title compound as a tan colored oil (5.9 g, 85%). MS (ESI): 252.2 (M+Na)⁺.

d) N-[2-[N-cyclopropyl-N-(2-methylpropyl)amino]thiazol-4-ylcarbonyl]-N′-[N-(7-methoxybenzofuran-2-ylcarbonyl)- L-b-cyclopropylalanyl]hydrazide

Following the procedure of Example 12(f)-12(g), except substituting (S)-2-(N-tert-butoxycarbonylamino)-3-cyclopropylpropionic acid for N-tert-butoxycarbonyl-L-leucine in step (f) and 7-methoxybenzofuran-2-carboxylic acid for 5-methyl-2-phenyloxazole-4-carboxylic acid in step (g), the title compound was prepared as a white solid (0.120 g, 74%). MS (ESI): 540.3 (M+H⁺).

### EXAMPLE 16

Preparation of 1-[N-(benzoxazol-5-ylcarbonyl)-L-leucinylamino]-3-[3-(2-pyridinyl)phenylacetylamino]propan-2-one

Following the procedure of Example 13(a)-13(g), except substituting benzoxazole-5-carboxylicacid for thiophene-2-carboxylic acid in step (f), the title compound was prepared. MS (ESI): 542 (M+H)⁺.

### EXAMPLE 17

Preparation of 1-[N-r4-[2-(N,N-dimethylamino)ethoxy-nzoyl]-L-leucinylamino ]-3-[3-(2-pyridinyl)phenylacetylamino]propan-2-one

Following the procedure of Example 13(a)-13(g), except substituting 4-[2-(N,N-dimethylamino)ethoxy]benzoic acid (J. Med. Chem. 27; 8; 1984; 1057-1066) for thiophene-2-carboxylic acid in step (f), the title compound was prepared. MS (ESI): 588 (M+H)⁺.

### EXAMPLE 18

Preparation of N-[2-FN-cyclopropyl-N-(2-methylproRyl)amino]thiazol-4-ylcarbonyl]-N′-[N-[5-r2-(N,N-dimethylamino)ethoxy]benzofuran-2-ylcarbonyl]- L-b-cyclopropylalanyl]hydrazide

a) ethyl 5-hydroxybenzofuran-2-carboxylate

To a mixture of aluminum chloride (6.3 g, 47.7 mmol) and ethanethiol (4.5 g, 72.9 mmol, 5.4 mL) at 0° C. was added ethyl 5-methoxybenzofuran-2-carboxylate (3.0 g, 13.6 mmol). After stirring at room temperature for 16 hours, the mixture was poured into water, acidified with 3N HCl, and extracted with dichloromethane (2x). The organic layers were combined, washed with brine, dried (MgSO₄), filtered and concentrated. The residue was purified by column chromatography (silica gel; ethyl acetate/hexanes) to yield the title compound as a white solid (2.2 g, 77%). ¹H NMR (400 MHz, CDCl₃) d 7.53 (s, 1H), 7.30-7.18 (m, 2H), 7.02 (d, 1H), 5.26 (s b, IH), 4.43 (q, 2H), 1.41 (t, 3H).

b) ethyl 5-[2(-N,N-dimethylamino)ethoxy]benzofuran-2-carboxylate

To a stirring solution of the compound of Example 18(a) (0.20 g, 0.971 mmol), N,N-dimethylethanolamine (0.122 g, 1.26 mmol, 0.127 mL), and triphenylphosphine (0.331 g, 1.26 mmol) in THF (3 mL) at 0C was added diisopropyl azodicarboxylate (0.254 g, 1.2 6 mmol, 0.248 mL) dropwise. After stirring at room temperature for 16 hours, the solution was concentrated and the residue purified by column chromatography (silica gel; ethyl acetate/hexanes) to yield the title compound as a white solid (0.161 g, 60%). MS (ESI): 278.2 (M+H⁺).

c) 5-[2(-N,N-dimethylamino)ethoxy]benzofuran-2-carboxylic acid

Following the procedure of Example 15(c), except substituting ethyl 5-[2(-N,N-dimethylamino)ethoxy]benzofuran-2-carboxylate for methyl (S)-2-(N-tert-butoxycarbonylamino)-3-cyclopropylpropionate, the title compound was prepared as a white solid (0.139 g, 96%). ¹H NMR (400 MHz, MeOH-d d 7.37 (m, 1H), 7.12 (m, 2H), 6.99 (m, 1H), 4.31 (t, 2H), 3.55 (t, 2H), 2.96 (s, 6H).

d) N-[2-[N-cyclopropyl-N-(2-methylpropyl)amino]thiazol-4-ylcarbonyl]-N′-[N-[5-[2-(N,N-dimethylamino)ethoxy]benzofuran-2-ylcarbonyl-]-L-b-cyclopropylalanyl]hydrazide

Following the procedure of Example 12(f)-12(g), except substituting (S)-2-(N-tert-butoxycarbonylamino)-3-cyclopropylpropionic acid for N-tert-butoxycarbonyl-L-leucine in step (f) and 5-[2(-N,N-dimethylamino)ethoxy]benzofuran-2-carboxylic acid for 5-methyl-2-phenyloxazole-4-carboxylic acid in step (g), the title compound was prepared as a white solid (0.131 g, 73%). MS (ESI): 597.3 (M+H⁺).

The above specification and Examples fully disclose how to make and use the compounds of the present invention. However, the present invention is not limited to the particular embodiments described hereinabove, but includes all modifications thereof within the scope of the following claims. The various references to journals, patents and other publications which are cited herein comprise the state of the art and are incorporated herein by reference as though fully set forth.

We claim:

1. Use of a compound selected from the group consisting of:
2-[N-(N-benzyloxycarbonylglycinyl)]-2′-[N′-(N-benzyloxycarbonyl-L-leucinyl)]carbohydrazide;
(3RS)-1-(N-benzyloxycarbonyl-L-leucinyl)-3-[N-(4-phenoxybenzoyl)amino]pyrrolidin-4-one;
(1S)-N-[2-[1-(N-benzyloxycarbonylamino)-3-methylbutyl]thiazol-4-ylcarbonyl]-N′-[N-(2-pyridinylmethoxycarbonyl)-L-leucinyl]hydrazide;
1-(N-benzyloxycarbonyl-L-leucinylamino)-3-(2-benzyloxyphenysulfonyl)amino-propan-2-one;
N-[2-(2-benzyloxyphenyl)thiazol-4-ylcarbonyl]-N′-[N-(3-pyridinylmethoxycarbonyl)-L-leucinyl]hydrazide;
(1S)-N-[2-[1-(N-benzyloxycarbonylamino)-3-methylbutyl]thiazol-4-ylcarbonyl]-N′-[N-(3-pyridinylmethoxycarbonyl)-L-leucinyl]hydrazide;
1-[N-(4-morpholinocarbamoyl)-L-leucinylamino]-3-(4-phenoxyphenylsulfonyl)amino-propan-2-one;
N-[2-(I-naphthyl)thiazol-4-ylcarbonyll-N′-[N-(4-pyridinylmethoxycarbonyl)-L-leucinyl]hydrazide;
N-[2-(2-benzyloxypbenyl)thiazol-4-ylcarbonyll-N-(N-pyrazinecarbonyl-L-leucinyl)hydrazide;
N-[N-( 1-benzyl-5-methylimidazol-4-ylcarbonyl)-L-leucinyl]-N′-[2-(1-naphthyl)thiazol-4-ylcarbonyl]hydrazide;
(3RS)-3-[N-(3-benzyloxybenzoyl)-L-leucinylamino]tetrahydrofuran-4-one;
N-[2-[N-cyclopropyl-N-(2-methylpropyl)amino]thiazol-4-ylcarbonyl]-N′-[N-(5-methyl-2-phenyloxazol-4-ylcarbonyl)-L-leucinyl]hydrazide;
1-[3-(2-pyridinyl)phenylacetylamino]-3-[N-(2-thiophenecarbonyl)-L-leucinylamino]propan-2-one;
(3S)-3-[N-(benzothiazol-6-ylcarbonyl)-L-leucinylamino]-1-[3-(2-pyridinyl)phenylacetylamino]butan-2-one;
N-[2-[N-cyclopropyl-N-(2-methylpropyl)amino]thiazol-4-ylcarbonyl]-N′-[N-(7-methoxybenzofuran-2-ylcarbonyl)- L-b-cyclopropylalanyl]hydrazide;
1-[N-(benzoxazol-5-ylcarbonyl)-L-leucinylamino]-3-[3-(2-pyridinyl)phenylacetylamino]propan-2-one;
1-[N-[4-[2-(N,N-dimethylamino)ethoxy]benzoyl]-L-leucinylamio]-3-[3-(2-pyridinyl)phenylacetylaminojpropan-2-one; and
N-[2-[N-cyclopropyl-N-(2-methylpropyl)amino]thiazol4-ylcarbonyll-N !-[N-[5-[2-(N,N-dimethylamino)ethoxy]benzofuran-2-ylcarbonyl]- L-β-cyclopropylalanyl]hydrazide, for use in the manufacture of a medicament for inhibiting a cysteine protease.

2. Use according to claim 1, wherein the cysteine protease is falcipain.

3. Use of a compound selected from the group consisting of:
2-[N-(N-benzyloxycarbonylglycinyl)]-2′-[N′-(N-benzyloxycarbonyl-L-leucinyl)]carbohydrazide;
(3RS)-1-(N-benzyloxycarbonyl-L-leucinyl)-3-[N-(4-phenoxybenzoyl)amino]pyrrolidin-4-one;
(1S)-N-[2-[1-(N-benzyloxycarbonylamino)-3-methylbutyl]thiazol-4-ylcarbonyl]-N′-[N-(2-pyridinylmethoxycarbonyl)-L-leucinyl]hydrazide;
1-(N-benzyloxycarbonyl-L-leucinylamino)-3-(2-benzyloxyphenylsulfonyl)amino-propan-2-one;
N-[2-(2-benzyloxyphenyl)thiazol-4-ylcarbonyl]-N′-[N-(3-pyridinylmethoxycarbonyl)-L-leucinyl]hydrazide;
(1S)-N-[2-[1-(N-benzyloxycarbonylamino)-3-methylbutyllthiazol-4-ylcarbonyl]-N′-[N-(3-pyridinyimethoxycarbonyl)-L-leucinyl]hydrazide;
1[N-(4-morpholinocarbamoyl)-L-leucinylamino]-3-(4-phenoxyphenylsulfonyl)amino-propan-2-one;
N-[2-(l-naphthyl)thiazol-4-ylcarbonyl]-N′N-(4-pyridinylmethoxycarbonyl)-L-leucinyl]hydrazide;
N-[2-(2-benzyloxyphenyl)thiazol-4-ylcarbonyl]-N′-(N-pyrazinecarbonyl-L-leucinyl)hydrazide;
N-[N-( I-benzyi-5-methylimidazol-4-ylcarbonyl)-L-leucinyl]-N′-[2-(1-naphthyl)thiazol-4-ylcarbonyl]hydrazide;
(3RS)-3-[N-(3-benzyloxybenzoyl)-L-leucinylaminoltetrahydrofuran-4-one;
N-[2-[N-cyclopropyl-N-(2-methylpropyl)amino]thiazol-4ylcarbonyl]-N′[N-(5-methyl-2-phenyloxazol-4-ylcarbonyl)-L-leucinyl]hydrazide;
1-[3-(2-pyridinyl)phenylacetylamino]-3-[N-(2-thiophenecarbonyl)-L-leucinylaminolpropan-2-one;
(3S)-3-[N-(benzothiazol-6-ylcarbonyl)-L-leucinylamino]-1-[3-(2-pyridinyl)phenylacetylamino]butan-2-one;
N-[2-[N-cyclopropyl-N-(2-methylpropyl)aminothiazol-4-ylcarbonyl]-N′N-(7-methoxybenzofuran-2-ylcarbonyl)-L-β-cyclopropylalanyl]hydrazide;
1-[N-(benzoxazol-5-ylcarbonyl)-L-leucinylamino]-3-[3-(2-pyridinyl)phenylacetylamino]propan-2-one;
1-[N-[4-[2-(N,N-dimethylamino)ethoxy]benzoyl]-L-leucinylamino]-3-[3-(2-pyridinyl)phenylacetylamino]propan-2-one; and
N-[2-[N-cyclopropyl-N-(2-methylpropylamino]thiazol-4-ylcarbonyl]-N′-[N-[5-[2-(N,N-dimethylamino)ethoxy]benzofuran-2-ylcarbonyl]-L-β-cyclopropylalanyl]hydrazide, for use in the manufacture of a medicament for treating a disease caused by infection by a parasite selected from the group consisting of: Plasmodium falciparuim, Trypanosoma cruzi, Trypanosoma Brucei, Leishmania mexicana, Leishmania pifanoi, Leishmania major, Schistosoma mansoni, Onchocerca volvulus, Brugia pahangi, Entamoeba histolytica, Giardia lamblia, the helminths Haemonchus contortus and Fasciola hepatica, the helminths of the genera Spirometra, Trichinella, Necator and Ascaris, and protozoa of the genera Cryptosporidium, Eimeria, Toxoplasma and Naegleria.

4. Use according to claim 2 wherein said disease is selected from a group consisting of: malaria, trypanosomiasis (African sleeping sickness, Chagas disease), leishmaniasis, schistosomiasis, onchocerciasis (river blindness) and giardiasis.

5. Use of a compound selected from the group consisting of:
2-[N-(N-benzyloxycarbonylglycinyl)]-2′-[N′-(N-benzyloxycarbonyl-L-leucinyl)]carbohydrazide;
(3RS)-1-(N-benzyloxycarbonyl-L-leucinyl)-3-[N-(4-phenoxybenzoyl)amino]pyrrolidin-4-one;
(1S)-N-[2-[1-(N-benzyloxycarbonylamino)-3-methylbutyl]thiazol-4-ylcarbonyl]-N′-[N-(2-pyridinylmethoxycarbonyl)-L-leucinyl]hydrazide;
1-(N-benzyioxycarbonyl-L-leucinylamino)-3-(2-benzyloxyphenyisulfonyl)amino-propan-2-one;
N-[2-(2-benzyloxyphenyl)thiazol-4-ylcarbonyl]-N′-[N-(3-pyridinylmethoxycarbonyl)-L-leucinyl]hydrazide;
(1S)-N-[2-[1-(N-benzyloxycarbonylamino)-3-methylbutyl]thiazol-4-ylcarbonyl]-N′-[N-(3-pyridinylmetboxycarbonyl)-L-leucinyl]hydrazide;
1-[N-(4-morpholinocarbamoyl)-L-leucinylamino]-3-(4-phenoxyphenylsulfonyl)amino-propan-2-one;
N-[2-( 1-naphthyl)thiazol-4-ylcarbonyl]-N′-[N-(4-pyridinylmethoxycarbonyl)-L-leucinyl]hydrazide;
N-[2-(2-benzyloxyphenyl)thiazol-4-ylcarbonyl]-N′-(N-pyrazinecarbonyl-L-leucinyl)hydrazide;
N-[N-(1-benzyl-5-methylimidazol-4-ylcarbonyl)-L-leucinyl]-N′-[2-(1-naphthyl)thiazol-4-ylcarbonyllhydrazide;
(3RS)-3-[N-(3-benzyloxybenzoyl)-L-leucinylamino]tetrahydrofuran-4-one;
N-[2-[N-cyclopropyl-N-(2-methylpropyl)amino]thiazol-4-ylcarbonyl]-N′-[N-(5-methyl-2-phenyloxazol-4ylcarbonyl)-L-leucinyl]hydrazide;
1-[3-(2-pyridinyl)phenylacetylamino]-3-[N-(2-thiophenecarbonyl)-L-leucinylamino]propan-2-one;
(3S)-3-[N-(benzothiazol-6-ylcarbonyl)-L-leucinylamino]-1-[3-(2-pyridinyl)phenylacetylaminoibutan-2-one;
N-[2-[N-cyclopropyl-N-(2-methylpropyl)amino]thiazol-4-ylcarbonyl]-N′-[N-(7-methoxybenzofuran-2-ylcarbonyl)- L-β-cyclopropylalanyl]hydrazide;
1-[N-(benzoxazol-5-ylcarbonyl)-L-leucinylaminol-3-[3-(2-pyridinyl)phenylacetylamino]propan-2-one;
1-[N-[4-[2-(N,N-dimethylamino)ethoxy]benzoyl]-L-leucinylamino]-3-[3-(2-pyridinyl)phenylacetylamino]propan-2-one; and
N-[2-[N-cyclopropyl-N-(2-methylpropyl)aminojthiazol-4-ylcarbonyll-N′-[N-[5-[2-(N,N-dimethylamino)etboxy]benzofuran-2-ylcarbonyl]- L-β-cyclopropylalanyl]hydrazide, for use in the manufacture of a medicament for treating malaria.

6. A pharmaceutical composition comprising a compound selected from the group consisting of:
2-[N-(N-benzyloxycarbonylglycinyl)]-2′-[N′-(N-benzyloxycarbonyl-L-leucinyl)]carbohydrazide;
(3RS)-1-(N-benzyloxycarbonyl-L-leucinyl)-3-[N-(4-phenoxybenzoyl)amino]pyrrolidin-4-one;
(1S)-N-[2-[1-(N-benzyloxycarbonylamino)-3-methylbutyl]thiazol-4-ylcarbonyl]-N′-[N-(2-pyridinylmethoxycarbonyl)-L-leucinyl]hydrazide;
1-(N-benzyoxycarbonyl-L-leucinylamino)-3-(2-benzyloxyphenylsulfonyl)amino-propan-2-one;
N-[2-(2-benzyloxyphenyl)thiazol-4-ylcarbonyl]-N′-[N-(3-pyridinylmethoxycarbonyl)-L-leucinyl]hydrazide;
(1S)-N-[2-[1-(N-benzyloxycarbonylamino)-3-methylbutyl]thiazol-4-ylcarbonyl]-N′-[N-(3-pyridinylmethoxycarbonyl)-L-leucinyl]hydrazide;
1-[N-(4morpholinocarbamoyl)-L-leucinylamino]-3-(4-phenoxyphenylsulfonyl)amino-propan-2-one;
N-[2-(]-naphthyI)thiazol-4-ylcarbonyl]-N′-[N-(4-pyridinylmethoxycarbonyl)-L-leucinyl]hydrazide;
N-[2-(2-benzyloxyphenyl)thiazol-4-ylcarbonyl]-N′-(N-pyrazinecarbonyt-L-leucinyl)hydrazide;
N-[N-(1-benzyl-5-methylimidazol-4-ylcarbonyl)-L-leucinyl]-N′-[2-(1-naphthyl)thiazol-4-ylcarbonyl]hydrazide;
(3RS)-3-[N-(3-benzyloxybenzoyl)-L-leucinylamino]tetrahydrofuran-4-one;
N-[2-[N-cyclopropyl-N-(2-methylpropyl)amino]thiazol-4-ylcarbonyl]-N′-[N-(5-methyl-2-phenyloxazol-4-ylcarbonyl)-L-leucinyl]hydrazide;
1-[3-(2-pyridinyl)phenylacetylamino]-3-[N-(2-thiophenecarbonyl)-L-leucinylamino]propan-2-one;
(3S)-3-[N-(benzothiazol-6-ylcarbonyl)-L-leucinylamino]1-[3-(2-pyridinyl)phenylacetylamino]butan-2-one;
N-[2-[N-cyclopropyl-N-(2-methylpropylamino]thiazol-4-ylcarbonyl]-N′-[N-(7-methoxybenzofuran-2-ylcarbonyl)- L-b-cyclopropylalanyl]hydrazide;
1-[N-(benzoxazol-5-ylcarbonyl)-L-leucinylamino]-3-[3-(2-pyridinyl)phenylacetylamino]propan-2-one;
1-[N-[4-[2-(N,N-dimethylamino)ethoxy]benzoyl]-L-leucinylamino]-3-[3-(2-pyridiny3)phenylacetylamino]propan-2-one; and
N-[2-[N-cyclopropyl-N-(2-methylpropyl)amino]thiazol-4-ylcarbonyl]-N′N-[5-[2-(N,N-dimethylamino)ethoxy]benzofuran-2-ylcarbonyl]-L-β-cyclopropylalanyl]hydrazide, and a pharmaceutically acceptable carrier, diluent or excipient.
